(19) United States
(12) Patent Application Publication
(10) Pub. No.: US 2025/0049972 A1
(43) Pub. Date: Feb. 13, 2025

(54) METHOD OF DETECTING PROSTATE CANCER IN A HUMAN SUBJECT USING A Cu 64 PSMA I&T INJECTION

(71) Applicant: Curium US LLC, St. Louis, MO (US)

(72) Inventors: Amanda Donovan, St. Louis, MO (US); Lauren Radford, St. Louis, MO (US); Allan Casciola, St. Louis, MO (US)

(21) Appl. No.: 18/798,723

(22) Filed: Aug. 8, 2024

Related U.S. Application Data
(60) Provisional application No. 63/518,272, filed on Aug. 8, 2023.
(60) Provisional application No. 63/588,202, filed on Oct. 5, 2023.
(60) Provisional application No. 63/621,965, filed on Jan. 17, 2024.
(60) Provisional application No. 63/626,838, filed on Jan. 30, 2024.

(51) Int. Cl. A61K 51/08

(52) U.S. Cl. CPC A61K 51/088; A61K 2123/00

(57) ABSTRACT

The present disclosure provides a method of detecting prostate cancer in a human subject wherein the human subject is administrated about 7 mCi to about 9 mCi of a Cu-64 PSMA I&T composition, images are obtained between 1 and 4 hours post administration, and wherein the images obtained retain acceptable image quality up to 4 hours post dose.

### CROSS-REFERENCE TO RELATED APPLICATION

This application claims priority to U.S. Provisional Application No. 63/518,272 filed on Aug. 8, 2023, U.S. Provisional Application No. 63/588,202 filed on Oct. 5, 2023, U.S. Provisional Application No. 63/621,965 filed on Jan. 17, 2024, and U.S. Provisional Application No. 63/626,838 filed on Jan. 30, 2024, all of which are hereby incorporated by reference in their entirety.

### FIELD OF THE INVENTION

The present disclosure relates to methods of using radiopharmaceutical compositions comprising copper that perform as a targeted molecular imaging agent for use in positron emission tomography (“PET”).

### BACKGROUND OF THE INVENTION

Prostate cancer (“PC”) is the second most common type of cancer among males in the world and the fifth most common cause of cancer-related mortality in males. Determination of serum prostate-specific antigen (“PSA”) levels is a standard procedure for screening and early detection of PC, but has demonstrated suboptimal diagnostic value. Although an improvement, noninvasive staging using standard imaging modalities, including computed tomography (“CT”), magnetic resonance imaging (“MRI”), and bone scintigraphy, provides unsatisfactory results with insufficient sensitivity in detecting remote and lymph node metastases (“LNMs”), thus leading to a significant underestimation of disease.

Different positron-emitting tracers like C-11 (carbon-11) choline and F-18 (fluorine-18) choline have been studied and found useful for the detection of lesions in PC patients using PET. C-11 or F-18 choline PET/CT imaging has been used to complement PSA levels for early detection of prostate adenocarcinoma; however, numerous clinical studies report low sensitivity and specificity, especially at low PSA levels and high Gleason scores. In addition, C-11 or F-18 choline imaging procedures are flow-limited, and higher uptake may occur in patients with benign prostatic hyperplasia. Although C-11 or F-18 choline PET/CT imaging has good specificity for detecting LNMs, this imaging procedure again shows low sensitivity, ranging from 10% to 73%.

Prostate-specific membrane antigen (“PSMA”), a membrane-bound type II glycoprotein with an extensive extracellular domain (44-750 amino acids), plays a significant role in prostate carcinogenesis and progression. This transmembrane protein is overexpressed in androgen-dependent and androgen-independent advanced metastatic prostate cancer, schwannoma, tumor neovasculature of many solid tumors, and in certain subtypes of bladder carcinoma, but shows low-level expression in normal prostate cells and organs such as the brain, kidneys, salivary glands, and small intestine. It is this collection of features that makes PSMA an exemplary target for PC diagnosis, therapy, and management.

PSMA-targeted PET/CT imaging has emerged as a highly sensitive method for detection of locally recurrent or metastatic lesions in the context of biochemical recurrence after primary prostate cancer treatment and for localization of primary prostate cancer. A number of gallium-68 (Ga-68)-labeled PSMA ligands have been evaluated clinically, and all appear similarly beneficial in the staging and management of patients with PC.

To date, Ga-68-labeled PSMA ligands have received the greatest attention in helping to manage patients with PC. However, the short half-life of Ga-68 (67.7 minutes) presents logistical limitations. This short half-life limits the application of Ga-68 PSMA to PET centers with nearby preparation capabilities using a germanium-68/gallium-68 generator and analytical groups to provide quality control for the final product.

A PET radionuclide with a longer half-life would provide an opportunity for PET diagnostic centers to utilize the diagnostic and patient management attributes of PSMA imaging. An excellent candidate for this application is copper-64 (Cu-64), which has a longer half-life of 12.7 hours and emits positrons of favorably low energy (Eβ⁺_{avg}=278 keV) compared to the average positron energy emitted by Ga-68 (Eβ⁺_{avg}=830 keV). Lower energy positrons travel shorter distances prior to annihilation, which can improve the resolution of a PET image since the resulting annihilation photons detected by the scanner are generated closer to their point of origin. The longer half-life of Cu-64 improves logistical constraints in both acceptable distances between the Cu-64 radiopharmaceutical manufacturing site and location of patient administration as well as increased flexibility in patient dosing schedules. Thus, the use of Cu-64 as a diagnostic radioisotope may enable delayed imaging time points, providing sufficient time for clearance from background tissues and resulting in increased image contrast and detection of smaller targeted areas.

In particular, providing sufficient and prolonged time for imaging in humans is not currently available and keenly sought after and desired by the medical industry. Given increased workloads, wait times, and stress on medical staff, providing an additional and prolonged window—to image human patients is in need in the industry. Indeed, increased imaging windows, without a significant decrease in image quality, allows for greater flexibility and more careful (and less rushed) analysis by medical staff. These benefits ultimately result in better care for human patients.

Thus, there exists a clinical need for an effective diagnostic agent for PC that can overcome the logistical constraints of shorter-lived PET agents, and that may offer improved sensitivity and resolution over similar Ga-68-labeled agents. There also exists a clinical need for an increased imaging window wherein medical staff can effectively and accurately image patients and then review the results within a longer time window, such as from 1 hour to 4 hours. The Cu-64 PSMA I&T injection disclosed herein is a new and promising diagnostic option for patients with PC and for allowing medical staff to image patients 1 to 4 hours post administration of the appropriate dose.

### SUMMARY OF THE INVENTION

Recent clinical studies in humans, as disclosed herein, show that the use of Cu-64 as a diagnostic radioisotope may enable delayed imaging time up to four times as long as originally expected, from approximately 1 to approximately 4 hours. Based on this unexpected result, disclosed herein is a method of diagnosing prostate cancer in the human body by administering a dose of Cu-64 PSMA I&T and then imaging the patient approximately 1 to approximately 4 hours post administration of the dose.

This disclosed method enables medical staff to effectively and accurately image patients up to 4.5 hours after administering the dose of Cu-64 PSMA I&T. This disclosed method thereby improves over the prior art by disclosing an increased imaging range from approximately 1 to approximately 4 hours while still concurrently allowing for accurate and effective imaging in humans. In other embodiments, the disclosed method provides an increased imaging range of approximately 45 minutes to approximately 4 hours and 30 minutes post administration of the Cu-64 PSMA I&T product while still producing accurate and effective imaging in humans.

In yet another embodiment, the method allows for the detection of prostate cancer in a patient in need thereof, comprising the steps of administering to the patient about 7 mCi to about 9 mCi of a Cu-64 PSMA I&T composition and imaging the patient between about 1 hour to about 4 hours post administration of the composition, wherein the image is of acceptable image quality.

In yet another embodiment, the method allows for the detection of prostate cancer in a patient in need thereof, comprising the steps of administering to the patient about 5 mCi to about 9 mCi of a Cu-64 PSMA I&T composition and imaging the patient between about 1 hour±15 minutes to about 4 hours±30 minutes post administration of the composition, wherein the image is of acceptable image quality.

In still another embodiment, the method allows for the detection of prostate cancer in a patient in need thereof, comprising the steps of administering to the patient about 5 mCi to about 9 mCi of a Cu-64 PSMA I&T composition and imaging the patient between about 1 hour to about 4 hours post administration of the composition, wherein the resulting image does not produce a correct detection rate below 80%.

Other features and aspects of the disclosure are described in detail below.

### BRIEF DESCRIPTION OF THE DRAWINGS

FIG. 1 is a general structure of Cu-64 PSMA I&T. Copper-64 is shown at the position where radiolabeling occurs.

FIG. 2 are images at 1 and 4 hours, respectively, of a patient injected with a dose of 8.89 mCi Copper 64 CU PSMA I&T compared to reference standard imaging.

FIG. 3 are images at 1 and 4 hours, respectively, of a patient injected with a dose of 8.46 mCi Copper 64 CU PSMA I&T compared to reference standard imaging.

FIG. 4 are images at 1 and 4 hours, respectively, of a patient injected with a dose of 8.59 mCi Copper 64 CU PSMA I&T compared to reference standard imaging.

FIG. 5 are images at 1 and 4 hours, respectively, of a patient injected with a dose of 8.34 mCi Copper 64 CU PSMA I&T compared to reference standard imaging.

FIG. 6 is the detection of metastatic pelvic lymph node with 8.9 mCi Copper 64 CU PSMA I&T on both 1 hour and 4 hours imaging confirmed by reference standard imaging in a 70-year old patient with PSA 0.6 ng/mL.

FIG. 7 is an independent reader assessment of general image quality of 1-hour scans based on number of lesions visible and image quality score of 5, 7 and 9 mCi scans (Mean [SD]).

FIG. 8 is an independent reader assessment of general image quality of 4-hour scans based on number of lesions visible and image quality score of 5, 7 and 9 mCi scans (Mean [SD]).

FIG. 9 is an independent reader assessment of lesion evaluation quality of 4-hour scans based on number of lesions visible and image quality score of 5, 7 and 9 mCi scans (Mean [SD]).

### DETAILED DESCRIPTION OF THE INVENTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents, and patent applications cited herein, whether supra or infra, are hereby incorporated by reference in their entirety.

Headings included herein are simply for ease of reference and are not intended to limit the disclosure in any way.

### I. Definitions

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present invention, particular methods and materials are now described.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

When introducing elements of the various embodiment(s) of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately,” when referring to a numerical value, shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors that may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Further, “about” or “approximately” may broaden a particular numerical value or range by at least ±10%. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

The term “Acceptable Image Quality” or “AIQ” as used herein, refers to image quality that is medically acceptable for diagnostic purposes.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “Correct Localization Rate” or “CLR” as used herein, refers to the percentage of regions containing at least one true PET positive lesion, with exactly localized correspondence between PET/CT imaging and the Composite Reference Standard regardless of any co-existent false positive findings, within the same region, out of all regions containing at least one PET positive finding.

The term “Correct Detection Rate” or “CDR” as used herein, refers to the percentage of patients who have at least one true PET positive lesion, with exactly localized correspondence between PET imaging and the Composite Reference Standard, regardless of any co-existent false positive findings, out of all patients who are scanned.

The term “Composite Reference Standard” as used herein, refers to the local histopathology obtained within 60 days before or following Cu-64 PSMA I&T PET/CT, or, if histopathology was not available, anatomical correlation of conventional image findings (e.g., targeted MRI or CT or any FDA-approved PET PSMA agent) obtained within 60 days before or following Cu-64 PSMA I&T PET/CT.

As used herein, the terms “end of synthesis”, “after formulation”, and “end of formulation” are used interchangeably to mean when the process of preparing the composition has completed. This may also include the time after quality control and release of the drug product by a qualified person.

The term “half-life,” as used herein in the chemical context, refers to the time it takes for half of the radioactive atoms of a specific radionuclide to decay.

The term “half-life” as used herein in the clinical context, refers to the biological half-life, for example, the time required for a drug's blood or plasma concentration to decrease by one half. This decrease in drug concentration is a reflection of its excretion or elimination after absorption is complete and distribution has reached an equilibrium or quasi equilibrium state. The half-life of a drug in the blood may be determined graphically off of a pharmacokinetic plot of a drug's blood-concentration time plot, typically after intravenous administration to a sample population. The half-life can also be determined using mathematical calculations that are well known in the art. Further, as used herein the term “half-life” also includes the “apparent half-life” of a drug. The apparent half-life may be a composite number that accounts for contributions from other processes besides elimination, such as absorption, reuptake, or enterohepatic recycling.

The terms “subject” or “patient” are used interchangeably herein and refer to a vertebrate, preferably a mammal. Mammals include, but are not limited to, humans.

### II. General Overview of the Copper 64 PSMA I&T Product

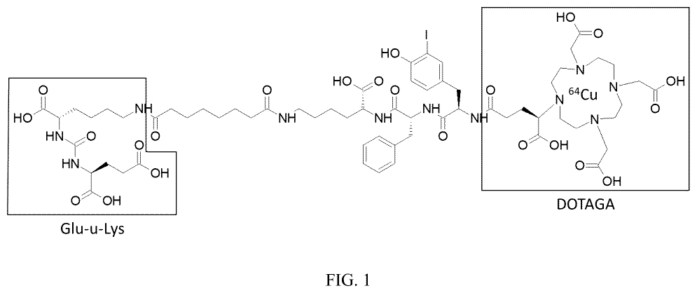

The present disclosure relates to a radiopharmaceutical composition comprising Cu-64 PSMA I&T, which combines the PET-imaging capabilities of Cu-64 with the PSMA-targeting capability of PSMA I&T to form a diagnostic agent capable of monitoring PSMA expression in vivo. Cu-64 PSMA I&T consists of the bioconjugate PSMA I&T radiolabeled with Cu-64, which is bound to PSMA I&T via the bifunctional chelator DOTAGA (See FIG. 1).

Cu-64 PSMA I&T injection is provided as a sterile-filtered radiopharmaceutical solution. The chemical name of Cu-64 PSMA I&T injection is: 64 Cu-(3S, 7S, 26R, 29R, 32R, 37R)-29-benzyl-32(4-hydroxy-3-iodobenzyl)-5, 13, 20, 28, 31, 34-hexaoxo-37-(4,7,10-tris(carboxymethyl)-1, 4, 7, 10-tetraazacyclododecan-1-yl)-4, 6, 12, 21, 27, 30, 33-heptaazaheptatriacontane-1, 3, 7, 26, 37-pentacarboxylic acid. It has the following chemical structure:

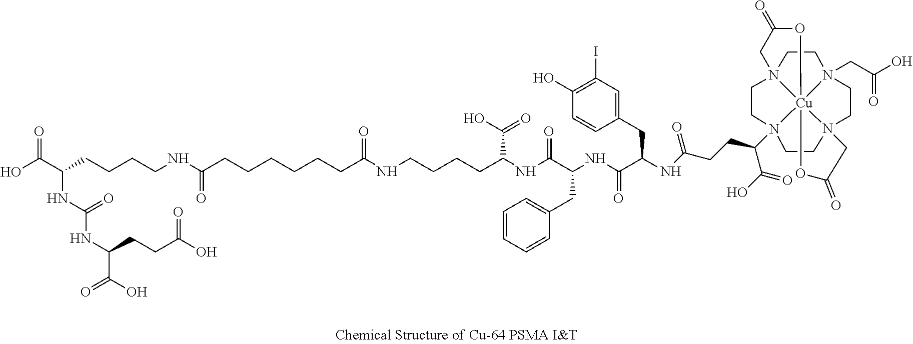

The invention disclosed herein offers several key advantages. The disclosed Cu-64 PSMA I&T formulations and methods presented herein allow for the preparation of higher activity batch sizes (e.g., up to 60 Ci or 2,220 GBq) and formulation of the final product as a chemically stable and ready-to-administer human dose. In another embodiment, the disclosed Cu-64 PSMA I&T preparation methods and formulations presented herein allow for the preparation of higher activity batch sizes of about 15 Ci to about 60 Ci or 555 GBq to about 2,220 GBq and formulation of the final product as a chemically stable and ready-to-administer human dose. In still another embodiment, the disclosed Cu-64 PSMA I&T preparation methods and formulations presented herein allow for the preparation of higher activity batch sizes of about 15 Ci, about 20 Ci, about 25 Ci, about 30 Ci, about 35 Ci, about 40 Ci, about 45 Ci, about 50 Ci, about 55 Ci, and about 60 Ci and formulation of the final product as a chemically stable and ready-to-administer human dose. In yet another embodiment, the disclosed Cu-64 PSMA I&T preparation methods and formulations presented herein allow for the preparation of batch sizes of about 250 mCi.

Cu-64 PSMA I&T injection is a PET-agent that specifically targets the prostate-specific membrane antigens that are expressed on metastatic prostate cancer cells. Specifically, Cu-64 PSMA I&T is indicated for the detection and localization of recurrent prostate cancer in males with biochemical recurrence based on elevated blood prostate-specific antigen (PSA) levels following prior treatment. Embodiments of the composition of the drug product are presented in Table 1(a) and (b).

TABLE 1(a)
A representative target composition of
a Copper Cu 64 PSMA I&T drug product

| | | | Quantity | Quantity |
|---|---|---|---|---|
| | | | per | per unit |
| Name of | | Quality | milliliter | dose |
| ingredient(s) | Function | standard | (1 mL) | (4 mL) |
| ⁶⁴Cu-PSMA I&T | Drug | GMP | 1.25-2.25 | 5-9 |
| | Substance/Active | | mCi^{a} | mCi^{a, b} |
| | Pharmaceutical | | | |
| | Ingredient | | | |
| Ascorbic Acid^{c} | Excipient (pH | USP | 32-39 | 84-148 |
| (Sodium Ascorbate) | Buffer and | | mg | mg^{b} |
| | Stabilizer) | | | |
| Sterile Water | Excipient | USP | q.s. | q.s. |
| for Injection | (Solvent) | | | |

q.s: quantum satis (as much as is enough)
^{a}At calibration.
^{b}The patient dose will be either 5 mCi, 7 mCi or 9 mCi;
^{c}Sodium ascorbate is used to formulate solutions for drug product manufacture. The concentration of sodium ascorbate is determined in the drug product and is listed in the specification as ascorbic acid content (target: 37 mg/mL).

TABLE 1(b)
Representative target compositions of
a Copper Cu 64 PSMA I&T drug product

| Name of ingredient(s) | Composition 1 | | Composition 2 | |
|---|---|---|---|---|
| Copper Cu-64 PSMA | 8 (+/−10%) mCi/vial | | 8 (+/−10%) mCi/vial | |
| I&T-Patient Dose | at calibration | | at injection time | |
| PSMA-I&T | ≤0.0028% | (w/v) | ≤0.0028% | (w/v) |
| Gentisic Acid | ≤0.06% | (w/v) | ≤0.06% | (w/v) |
| Ascorbic Acid | 3.2-4.2% | (w/v) | 3.2-4.2% | (w/v) |
| Sterile Water | 100% | (v/v) | 100% | (v/v) |
| for Injection | | | | |

### III. Additional Composition and Formulations Details

In one embodiment, the medicinal product or radiopharmaceutical composition (or formulation) is a sterile-filtered radiopharmaceutical solution containing a dose of Cu-64 PSMA I&T in an aqueous sodium acetate/gentisic acid solution containing sodium ascorbate/ascorbic acid. The product is diluted to a standard concentration, and therefore, the final volume of the bulk product varies depending on the starting activity introduced.

In another embodiment, the radiopharmaceutical composition or formulation comprises at least one stabilizing agent, a pH adjuster, a metal ion chelator, or a combination thereof.

In another embodiment, one or more stabilizers are selected from the list comprising ethanol, para-aminobenzoic acid (PABA), dihydroxybenzoic acid (gentisate compounds), gentisic acid, cysteine, selenomethionine, ascorbic acid/sodium ascorbate, and methionine.

In another embodiment, pH adjusters are selected from the group consisting of sodium acetate/acetic acid, gentisic acid, ascorbic acid/sodium ascorbate, ammonium acetate, citric acid, sodium citrate, sodium gentisic, and sodium carbonate/bicarbonate.

In one embodiment, the radiopharmaceutical composition or formulation has a purity of at least about 90%, at least about 95%, at least about 96%, at least about 97%, at least about 98%, or at least about 99% as measured by high-performance liquid chromatography (“HPLC”), thin-layer chromatography (“TLC”), instant thin-layer chromatography (“iTLC”), or gas chromatography (“GC”). In another embodiment, the radiopharmaceutical composition or formulation has a purity of about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, or about 99.5% as measured by HPLC, TLC, iTLC, or GC.

In one embodiment, the radiopharmaceutical composition or formulation is stored at a temperature from about 2° C. to about 55° C., from about 10° C. to about 50° C., from about 15° C. to about 45° C., or from about 20° C. to about 40° C. In one specific embodiment, the radiopharmaceutical composition or formulation is stored at a temperature at about 10° C., about 12.5° C., about 15° C., about 17.5° C., about 20° C., about 20.5° C., about 21° C., about 21.5° C., about 22° C., about 22.5° C., about 23° C., about 23.5° C., about 24° C., about 24.5° C., about 25° C., about 25.5° C., about 26° C., about 26.5° C., about 27° C., about 27.5° C., about 28° C., about 28.5° C., about 29° C., about 29.5° C., about 30° C., about 32.5° C., about 35° C., about 37.5° C., about 40° C., about 42.5° C., about 45° C., about 47.5° C., about 50° C., about 52.5° C., or about 55° C.

In one embodiment, the unit dose is 1 mL, 2 mL, 3 mL, 4 mL, 5 mL, 6 mL, 7 mL, 8 mL, 9 mL, or 10 mL. In another embodiment, the unit dose is 3.0 mL, 3.1 mL, 3.2 mL, 3.3 mL, 3.4 mL, 3.5 mL, 3.6 mL, 3.7 mL, 3.8 mL, 3.9 mL, or 4 mL. In a specific embodiment, the unit dose is 4 mL. In a further embodiment, the unit dose is 3.6 mL.

### A. Cu-64 PSMA I&T

The total amount of labeled Cu-64 PSMA I&T present in the radiopharmaceutical composition can and will vary. In some embodiments, the total amount of Cu-64 PSMA I&T present in the radiopharmaceutical composition may be up to about 50 mCi/mL at end of synthesis (“EOS”). In various embodiments, the total amount of Cu-64 PSMA I&T present in the radiopharmaceutical composition may be about 5 mCi/mL, about 7.5 mCi/mL, about 10 mCi/mL, about 12.5 mCi/mL, about 15 mCi/mL, about 17.5, about 20 mCi/mL, about 22.5 mCi/mL, about 25 mCi/mL, about 27.5 mCi/mL, about 30 mCi/mL, about 32.5 mCi/mL, about 35 mCi/mL, about 37.5 mCi/mL, about 40 mCi/mL, about 42.5 mCi/mL, about 45 mCi/mL, about 47.5 mCi/mL, or about 50 mCi/mL at EOS. In still other embodiments, the total amount of Cu-64 PSMA I&T present in the radiopharmaceutical composition may be about 30 mCi/mL, about 31 mCi/mL, about 32 mCi/mL, about 33 mCi/mL, about 34 mCi/mL, about 35, about 36 mCi/mL, about 37 mCi/mL, about 38 mCi/mL, about 39 mCi/mL, or about 40 mCi/mL at EOS. In another embodiment, the total amount of Cu-64 PSMA I&T present in the radiopharmaceutical composition is about 25 mCi/mL to about 50 mCi/mL at EOS. In yet another embodiment, the total amount of Cu-64 PSMA I&T present in the radiopharmaceutical composition is about 30 mCi/mL to about 45 mCi/mL at EOS. In yet another embodiment, the total amount of Cu-64 PSMA I&T present in the radiopharmaceutical composition is about 35 mCi/mL to about 40 mCi/mL at EOS.

In another embodiment, the total amount of labeled Cu-64 PSMA I&T present in the radiopharmaceutical composition may range from about 1 mCi/mL-3 mCi/mL at calibration. In various embodiments, the total amount of labeled Cu-64 PSMA I&T present at calibration may be about 1 mCi/mL, about 1.25 mCi/mL, about 1.5 mCi/mL, about 1.75 mCi/mL, about 2 mCi/mL, about 2.25 mCi/mL, about 2.5 mCi/mL, 2.75 mCi/mL, or about 3 mCi/mL.

The amount of PSMA I&T present in the radiopharmaceutical composition can also vary. In one embodiment, the amount PSMA I&T present in the radiopharmaceutical composition may range from about 10 μg/mL to about 30 μg/mL PSMA I&T. In various embodiments, the total amount of PSMA I&T may be about 1 μg/mL, about 1.25 μg/mL, about 1.5 μg/mL, about 1.75 μg/mL, about 2 μg/mL, about 2.25 μg/mL, or about 2.5 μg/mL. In another embodiment, the total amount of PSMA I&T may range from 1 μg/mL to about 30 μg/mL, from about 10 μg/mL to about 30 μg/mL, from about 15 μg/mL to about 27.5 μg/mL, or from about 20 μg/mL to about 25 μg/mL. In another embodiment, the total amount of PSMA I&T may range from about 22.5 μg/mL to about 27.5 μg/mL.

In one embodiment, the radioactivity of the Cu-64 PSMA I&T in the radiopharmaceutical composition at EOS is less than about 150 mCi, less than about 140 mCi, less than about 130 mCi, less than about 120 mCi, less than about 110 mCi, less than about 100 mCi, less than about 90 mCi, less than about 80 mCi, less than about 70 mCi, less than about 60 mCi, less than about 50 mCi, less than about 40 mCi, less than about 30 mCi, less than about 20 mCi, or less than about 10 mCi per unit dose. In another embodiment, the radioactivity of the Cu-64 PSMA I&T in the radiopharmaceutical composition at EOS is from about 1 mCi to about 150 mCi, from about 1 mCi to about 125 mCi, from about 1 mCi to about 100 mCi, from about 1 mCi to about 75 mCi, from about 1 mCi to about 50 mCi, from about 1 mCi to about 40 mCi, from about 1 mCi to about 30 mCi, from about 1 mCi to about 20 mCi, from about 1 mCi to about 10 mCi, or from about 4 mCi to about 10 mCi, or from about 5 mCi to about 9 mCi, per unit dose. In one specific embodiment, the radioactivity of the Cu-64 PSMA I&T in the radiopharmaceutical composition is about 1 mCi, about 2 mCi, about 3 mCi, about 4 mCi, about 5 mCi, about 6 mCi, about 7 mCi, about 8 mCi, about 9 mCi, about 10 mCi, about 11 mCi, about 12 mCi, about 13 mCi, about 14 mCi, about 15 mCi, about 18 mCi, about 21 mCi, about 24 mCi, about 27 mCi, about 30 mCi, about 40 mCi, about 50 mCi, about 60 mCi, about 70 mCi, about 80 mCi, about 90 mCi, about 100 mCi, about 110 mCi, about 120 mCi, about 130 mCi, about 140 mCi, or about 150 mCi per unit dose.

In another embodiment, the radioactivity of the Cu-64 PSMA I&T in the radiopharmaceutical composition at EOS is less than about 50 mCi, less than about 40 mCi, less than about 30 mCi, less than about 20 mCi, less than about 10 mCi, less than about 5 mCi, less than about 4 mCi, less than about 3 mCi, less than about 2.5 mCi, less than about 2 mCi, less than about 1.5 mCi, or less than about 1 mCi per 1 mL. In another embodiment, the radioactivity of the Cu-64 PSMA I&T in the radiopharmaceutical composition is from about 1 mCi to about 100 mCi, from about 1 mCi to about 50 mCi, from about 1 mCi to about 20 mCi, or from about 1 mCi to about 10 mCi per 1 mL. In one specific embodiment, the radioactivity of the Cu-64 PSMA I&T in the radiopharmaceutical composition at EOS is about 1 mCi, about 1.25 mCi, about 1.5 mCi, about 1.75 mCi, about 2 mCi, about 2.25 mCi, about 2.5 mCi, about 2.75 mCi, about 3 mCi, about 3.25 mCi, about 3.5 mCi, about 3.75 mCi, about 4 mCi, about 4.25 mCi, about 4.5 mCi, about 4.75 mCi, about 5 mCi, about 7 mCi, about 9 mCi, about 12 mCi, about 15 mCi, about 18 mCi, about 21 mCi, about 24 mCi, about 27 mCi, about 30 mCi, about 40 mCi, or about 50 mCi per 1 mL.

In one embodiment, the radioactivity of the Cu-64 PSMA I&T in the radiopharmaceutical composition at the time of calibration is less than about 150 mCi, less than about 140 mCi, less than about 130 mCi, less than about 120 mCi, less than about 110 mCi, less than about 100 mCi, less than about 90 mCi, less than about 80 mCi, less than about 70 mCi, less than about 60 mCi, less than about 50 mCi, less than about 40 mCi, less than about 30 mCi, less than about 20 mCi, or less than about 10 mCi per unit dose. In another embodiment, the radioactivity of the Cu-64 PSMA I&T in the radiopharmaceutical composition at the time of calibration is from about 1 mCi to about 150 mCi, from about 1 mCi to about 125 mCi, from about 1 mCi to about 100 mCi, from about 1 mCi to about 75 mCi, from about 1 mCi to about 50 mCi, from about 1 mCi to about 40 mCi, from about 1 mCi to about 30 mCi, from about 1 mCi to about 20 mCi, from about 1 mCi to about 10 mCi, or from about 4 mCi to about 10 mCi, or from about 5 mCi to about 9 mCi, per unit dose. In one specific embodiment, the radioactivity of the Cu-64 PSMA I&T in the radiopharmaceutical composition at the time of calibration is about 1 mCi, about 2 mCi, about 3 mCi, about 4 mCi, about 5 mCi, about 6 mCi, about 7 mCi, about 8 mCi, about 9 mCi, about 10 mCi, about 11 mCi, about 12 mCi, about 13 mCi, about 14 mCi, about 15 mCi, about 18 mCi, about 21 mCi, about 24 mCi, about 27 mCi, about 30 mCi, about 40 mCi, about 50 mCi, about 60 mCi, about 70 mCi, about 80 mCi, about 90 mCi, about 100 mCi, about 110 mCi, about 120 mCi, about 130 mCi, about 140 mCi, or about 150 mCi per unit dose.

In another embodiment, the radioactivity of the Cu-64 PSMA I&T in the radiopharmaceutical composition at the time of calibration is less than about 50 mCi, less than about 40 mCi, less than about 30 mCi, less than about 20 mCi, less than about 10 mCi, less than about 5 mCi, less than about 4 mCi, less than about 3 mCi, less than about 2.5 mCi, less than about 2 mCi, less than about 1.5 mCi, or less than about 1 mCi per 1 mL. In another embodiment, the radioactivity of the Cu-64 PSMA I&T in the radiopharmaceutical composition at the time of calibration is from about 1 mCi to about 100 mCi, from about 1 mCi to about 50 mCi, from about 1 mCi to about 20 mCi, or from about 1 mCi to about 10 mCi per 1 mL. In one specific embodiment, the radioactivity of the Cu-64 PSMA I&T in the radiopharmaceutical composition at the time of calibration is about 1 mCi, about 1.25 mCi, about 1.5 mCi, about 1.75 mCi, about 2 mCi, about 2.25 mCi, about 2.5 mCi, about 2.75 mCi, about 3 mCi, about 3.25 mCi, about 3.5 mCi, about 3.75 mCi, about 4 mCi, about 4.25 mCi, about 4.5 mCi, about 4.75 mCi, about 5 mCi, about 7 mCi, about 9 mCi, about 12 mCi, about 15 mCi, about 18 mCi, about 21 mCi, about 24 mCi, about 27 mCi, about 30 mCi, about 40 mCi, or about 50 mCi per 1 mL.

In one embodiment, the RAC of the Cu-64 PSMA I&T in the radiopharmaceutical composition is less than about 50 mCi/mL, less than about 45 mCi/mL, less than about 40 mCi/mL, less than about 35 mCi/mL, less than about 30 mCi/mL, less than about 25 mCi/mL, less than about 20 mCi/mL, less than about 15 mCi/mL, less than about 10 mCi/mL, less than about 5 mCi/mL, less than about 4 mCi/mL, less than about 3 mCi/mL, or less than about 2 mCi/mL.

In still another embodiment, the RAC of the Cu-64 PSMA I&T in the radiopharmaceutical composition is up to about 40 mCi/mL, up to about 39 mCi/mL, up to about 38 mCi/mL, up to about 37 mCi/mL, up to about 36 mCi/mL, up to about 35 mCi/mL, up to about 34 mCi/mL, up to about 33 mCi/mL, up to about 32 mCi/mL, up to about 31 mCi/mL, up to about 30 mCi/mL, up to about 29 mCi/mL, up to about 28 mCi/mL, up to about 27 mCi/mL, up to about 26 mCi/mL, up to about 25 mCi/mL, up to about 24 mCi/mL, up to about 23 mCi/mL, up to about 22 mCi/mL, up to about 21 mCi/mL, up to about 20 mCi/mL, up to about 19 mCi/mL, up to about 18 mCi/mL, up to about 17 mCi/mL, up to about 16 mCi/mL, up to about 15 mCi/mL, 14 mCi/mL, up to about 13 mCi/mL, up to about 12 mCi/mL, up to about 11 mCi/mL, up to about 10 mCi/mL, up to about 9 mCi/mL, up to about 8 mCi/mL, up to about 7 mCi/mL, up to about 6 mCi/mL, up to about 5 mCi/mL, up to about 4 mCi/mL, up to about 3 mCi/mL, up to about 2 mCi/mL, or up to about 1 mCi/mL.

In another embodiment, the RAC of the Cu-64 PSMA I&T in the radiopharmaceutical composition is from about 1 mCi/mL to about 100 mCi/mL, about 2 mCi/mL to about 90 mCi/mL, about 3 mCi/mL to about 80 mCi/mL, about 4 mCi/mL to about 60 mCi/mL, about 5 mCi/mL to about 50 mCi/mL, from about 10 mCi/mL to about 40 mCi/mL, about 1 mCi/mL to about 40 mCi/mL, about 1 mCi/mL to about 36 mCi/mL, about 1 mCi/mL to about 35 mCi/mL, or about 20 mCi/mL to about 35 mCi/mL. In one specific embodiment, the RAC of the Cu-64 PSMA I&T in the radiopharmaceutical composition is about 1 mCi/mL, about 10 mCi/mL, about 13.5 mCi/mL, about 15 mCi/mL, about 20 mCi/mL, about 27 mCi/mL, about 30 mCi/mL, about 33 mCi/mL, about 35 mCi/mL, about 40 mCi/mL, about 45 mCi/mL, or about 50 mCi/mL.

In yet another embodiment, the Cu-64 PSMA I&T drug product has a standard concentration at the end of production of about 15 mCi/mL to about 40 mCi/mL at the end of production.

In one embodiment, the mass of radioactive pharmaceutical ingredient (Cu-64 PSMA I&T) in the drug product and related non-radioactive substances is less than about 200 μg, less than about 175 μg, less than about 150 μg, less than about 125 μg, less than about 120 μg, less than about 115 μg, less than about 110 μg, less than about 105 μg, less than about 100 μg, less than about 75 μg, or less than about 50 μg per vial. In another embodiment, the mass of radioactive pharmaceutical ingredient (Cu-64 PSMA I&T) in the drug product and related non-radioactive substances is less than about 100 μg, less than about 90 μg, less than about 80 μg, less than about 70 μg, less than about 60 μg, less than about 50 μg, less than about 40 μg, less than about 30 μg, less than about 20 μg, less than about 10 μg per vial. In still another embodiment, the mass of radioactive pharmaceutical ingredient (Cu-64 PSMA I&T) in the drug product and related non-radioactive substances is less than about 120 μg. In a specific embodiment, the mass of radioactive pharmaceutical ingredient (Cu-64 PSMA I&T) in the drug product and related non-radioactive substances is less than or equal to about 100 μg.

### B. Antioxidants

In one embodiment, the formulation comprises an antioxidant. In another embodiment, the antioxidant is ascorbate/ascorbic acid. In another embodiment, the antioxidant is gentisic acid. In one specific embodiment, the antioxidant is sodium acetate/gentisic acid solution.

In another embodiment, the total amount of sodium acetate/gentisic acid solution in the radiopharmaceutical composition can and will vary. In some embodiments, sodium acetate/gentisic acid solution present in the radiopharmaceutical composition may range from about 0 μg/mL to about 600 μg/mL or from about 130 μg/mL to about 320 μg/mL of gentisic acid, and about 0 mg/mL to about 3.5 mg/mL or from about 1.3 mg/mL to about 3.3 mg/mL of sodium acetate.

In another embodiment, the concentration of sodium acetate in the final radiopharmaceutical composition may be from about 0.2 M to about 0.5 M, from about 0.25 M to about 0.4 M, or from about 0.3 M to about 0.35 M.

In yet another embodiment, the concentration of sodium acetate in the final radiopharmaceutical composition may be from about 1 mg/mL to about 5 mg/mL, from about 2 mg/mL to about 4 mg/mL, or from about 2.5 mg/mL to about 3.5 mg/mL. In another embodiment, the concentration of sodium acetate in the final radiopharmaceutical composition may be from about 0 mg/mL to about 1 mg/mL. In still another embodiment, the concentration of sodium acetate in the final radiopharmaceutical composition may be less than or equal to about 1 mg/mL.

In the final radiopharmaceutical composition dose, the concentration of gentisic acid may range from about 0 mg/mL to about 3.5 mg/mL. In another embodiment, the concentration of gentisic acid may range from about 1.3 mg/mL to about 3.3 μg/mL.

In the final radiopharmaceutical composition dose, the concentration of gentisic acid may range from about 0 mg/mL to about 600 μg/mL. In another embodiment, the concentration of gentisic acid may range from about 130 mg/mL to about 320 μg/mL.

### C. Stabilizing Agent

In one embodiment, the formulation comprises stabilizing agent.

In another embodiment, the stabilizing agent is sodium ascorbate. The total amount of sodium ascorbate in the radiopharmaceutical composition can and will vary. In some embodiments, sodium ascorbate present in the radiopharmaceutical composition may range from about 5 mg to about 300 mg per unit dose. In one embodiment, sodium ascorbate is present in the radiopharmaceutical composition in an amount of about 10 mg, about 20 mg, about 30 mg, about 40 mg, about 50 mg, about 60 mg, about 70 mg, or about 75 mg per 1 mL. In still another embodiment, sodium ascorbate is present in the radiopharmaceutical composition in an amount of about 30 mg, about 31 mg, about 32 mg, about 33 mg, about 34 mg, about 35 mg, about 36 mg, about 37 mg, about 38 mg, about 39 mg or about 40 mg per 1 mL. In yet another embodiment, sodium ascorbate is present in the radiopharmaceutical composition in an amount from about 1 mg to about 500 mg, from about 5 mg to about 300 mg, from about 20 mg to about 80 mg, from about 30 mg to about 60 mg, from about 35 mg to about 50 mg, or from about 40 mg to about 45 mg per 1 mL.

One aspect of the disclosure provides for a radiopharmaceutical composition with a pH from about 3 to about 9, from about 4 to about 9, from about 5 to about 9, from about 3 to about 8, from about 4 to about 8, from about 3 to about 7.5, from about 5 to about 7.5, or from about 5.5 to about 7.5. The pH of the radiopharmaceutical composition may be about 3.0, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

### D. Ligand

The present invention includes ligands that are capable of being chelated to Cu-64. In one embodiment, the ligand is PSMA I&T.

The amount of ligand that is added to a reaction mixture containing Cu-64 varies depending on the amount of activity of Cu-64 present. For example, Table 2 shows varying amounts of ligand, PSMA, added to the reaction mixture depending on the range of Cu-64 present at the time of synthesis.

TABLE 2

PSMA Range
(μg)

| Cu-64 Range | Cu-64 | Cu-64 |
|---|---|---|
| (mCi at | mCi × | mCi × |
| synthesis) | 0.4 μg/mCi | 0.6 μg/mCi |
| 200 | 80 | 120 |
| 500 | 200 | 300 |
| 800 | 320 | 480 |
| 1000 | 400 | 600 |
| 3000 | 1200 | 1800 |
| 5000 | 2000 | 3000 |
| 7000 | 2800 | 4200 |
| 9000 | 3600 | 5400 |
| 12000 | 4800 | 7200 |
| 15000 | 6000 | 9000 |
| 20000 | 8000 | 12000 |

In one embodiment where the reaction mixture contains about 15 Ci Cu-64, the ligand is added to the reaction mixture in an amount from about 8,000 μg to about 10,000 μg. In another embodiment, about 9,000 μg is added to the reaction mixture.

In another embodiment where the reaction mixture contains about 15 Ci Cu-64, the ligand is added to the reaction mixture in an amount from about 7,000 μg to about 11,000 μg, from about 8,000 μg to about 10,000 μg, or about 9,000 μg is added to the reaction mixture.

In another embodiment where the reaction mixture contains about 20 Ci Cu-64, the ligand is added to the reaction mixture in an amount from about 8,000 μg to about 12,000 μg, from about 9,000 μg to about 11,000 μg, or about 10,000 μg is added to the reaction mixture.

In another embodiment where the reaction mixture contains about 50 Ci Cu-64, the ligand is added to the reaction mixture in an amount from about 20,000 μg to about 30,000 μg, from about 22,000 μg to about 28,000 μg, from about 24,000 μg to about 26,000 μg, or about 25,000 μg is added to the reaction mixture.

In still another embodiment where the reaction mixture contains about 90 Ci Cu-64, the ligand is added to the reaction mixture in an amount from about 36,000 μg to about 54,000 μg, from about 40,000 μg to about 50,000 μg, or about 45,000 μg is added to the reaction mixture.

In another embodiment, PSMA I&T is added to the reaction mixture in an amount from about 1,000 μg to about 60,000 μg, from about 5,000 μg to about 55,000 μg, from about 7,000 μg to about 11,000 μg, from about 8,000 μg to about 12,000 μg, from about 8,000 μg to about 10,000 μg, from about 9,000 μg to about 11,000 μg, from about 20,000 μg to about 30,000 μg, from about 22,000 μg to about 28,000 μg, from about 24,000 μg to about 26,000 μg, from about 36,000 μg to about 54,000 μg, or from about 40,000 μg to about 50,000 μg. In another embodiment, PSMA I&T is added to the reaction mixture in an amount of about 9,000 μg, about 10,000 μg, about 25,000 μg, or about 45,000 μg. In yet another embodiment, the PSMA I&T is added to the reaction mixture in an amount of less than about 11,000 μg, less than about 30,000 μg, or less than about 55,000 μg.

In a further embodiment, PSMA I&T is added to the reaction mixture in an amount of about 9,000 μg for about a 15 Ci batch. In another embodiment, PSMA I&T is added to the reaction mixture in an amount of about 8,000 μg, of about 9,000 μg, of about 10,000 μg, or of about 11,000 μg for about a 20 Ci batch. In still another embodiment, PSMA I&T is added to the reaction mixture in an amount of about 8,000 μg to about 11,000 μg for about a 20 Ci batch. In yet another embodiment, PSMA I&T is added to the reaction mixture in an amount of about 20,000 μg to about 30,000 μg for about a 50 Ci batch. In still another embodiment, PSMA I&T is added to the reaction mixture in an amount of about 36,000 μg to about 54,000 μg for about a 90 Ci batch.

In another embodiment, PSMA I&T is used in an amount from about 0.1 μg/mCi to about 20 μg/mCi, from about 0.5 μg/mCi to about 15 μg/mCi, from about 1 μg/mCi to about 11 μg/mCi, from about 1 μg/mCi to about 8 μg/mCi, from about 1 μg/mCi to about 5 μg/mCi, from about 1 μg/mCi to about 3 μg/mCi, or from about 0.1 μg/mCi to about 1.5 μg/mCi. In yet another embodiment, the PSMA I&T is used in an amount of about 0.1 μg/mCi, about 0.25 μg/mCi, about 0.4 μg/mCi, about 0.5 μg/mCi, about 0.6 μg/mCi, about 0.75 μg/mCi, about 0.8 μg/mCi, about 1 μg/mCi, about 1.25 μg/mCi, about 1.5 μg/mCi, about 1.75 μg/mCi, about 2 μg/mCi, about 2.5 μg/mCi, about 3 μg/mCi, about 3.5, or about 4 μg/mCi.

In one embodiment, the concentration of PSMA I&T per mL in the radiolabeling step is greater than about 100 μg/mL, greater than about 150 μg/mL, greater than about 200 μg/mL, greater than about 250 μg/mL, greater than about 300 μg/mL, greater than about 333 μg/mL, or greater than about 400 μg/mL. In still another embodiment, the concentration of PSMA I&T per mL in the radiolabeling step is greater than about 100 μg/mL, greater than about 110 μg/mL, greater than about 120 μg/mL, greater than about 130 μg/mL, greater than about 140 μg/mL, greater than about 150 μg/mL, greater than about 160 μg/mL, greater than about 170 μg/mL, greater than about 180 μg/mL, greater than about 190 μg/mL, or greater than about 200 μg/mL.

### E. Radionuclide

In one embodiment, ⁶⁴CuCl₂ is added to the reaction mixture (as a source of ⁶⁴Cu) in an amount from about 100 mCi to about 5000 mCi, from about 200 mCi to about 4000 mCi, from about 300 mCi to about 3500 mCi, from about 400 mCi to about 3000 mCi, from about 500 mCi to about 2500 mCi, from about 500 mCi to about 15,000 mCi, from about 1,000 mCi to about 10,000 mCi, from about 1,000 mCi to about 15,000 mCi, or from about 7,500 mCi to about 15,000 mCi. In another embodiment, ⁶⁴CuCl₂ is added to the reaction mixture (as a source of ⁶⁴Cu) in an amount up to about 10,000 mCi. In yet another embodiment, ⁶⁴CuCl₂ is added to the reaction mixture (as a source of ⁶⁴Cu) in an amount up to about 15,000 mCi. In a further embodiment, ⁶⁴CuCl₂ is added to the reaction mixture (as a source of ⁶⁴Cu) in an amount of about 100 mCi, about 200 mCi, about 300 mCi, about 400 mCi, about 500 mCi, about 600 mCi, about 700 mCi, about 800 mCi, about 900 mCi, about 1000 mCi, about 1500 mCi, about 2000 mCi, about 2500 mCi, about 3000 mCi, about 3500 mCi, about 4000 mCi, about 4500 mCi, about 5000 mCi, about 5500 mCi, about 6000 mCi, about 6500 mCi, about 7000 mCi, about 7500 mCi, about 8000 mCi, about 8500 mCi, about 9000 mCi, about 9500 mCi, about 10,000 mCi, about 15,000 mCi, about 20,000 mCi, about 30,000 mCi, about 40,000 mCi, about 50,000 mCi, about 60,000 mCi, about 70,000 mCi, about 80,000 mCi, about 90,000 mCi, or about 100,000 mCi. In yet another embodiment, ⁶⁴CuCl₂ is added to the reaction mixture (as a source of ⁶⁴Cu) in an amount of less than about 100 mCi, less than about 200 mCi, less than about 300 mCi, less than about 400 mCi, less than about 500 mCi, less than about 600 mCi, less than about 700 mCi, less than about 800 mCi, less than about 900 mCi, less than about 1000 mCi, less than about 1500 mCi, less than about 2000 mCi, less than about 2500 mCi, less than about 3000 mCi, less than about 3500 mCi, less than about 4000 mCi, less than about 4500 mCi, less than about 5000 mCi, less than about 5500 mCi, less than about 6000 mCi, less than about 6500 mCi, less than about 7000 mCi, less than about 7500 mCi, less than about 8000 mCi, less than about 8500 mCi, less than about 9000 mCi, less than about 9500 mCi, or less than about 10,000 mCi.

In another embodiment, non-carrier copper, such as CuCl₂, is added to the reaction mixture to improve the consistency of the radiochemical purity of the precursor formulation. This process is referred to herein as “spiking.” In one embodiment, there is at least about 5 ppm of non-carrier copper in the precursor formulation. In another embodiment, there is at least about 1 ppm, about 2 ppm, about 3 ppm, about 4 ppm, about 5 ppm, about 10 ppm, about 15 ppm, about 20 ppm, about 25 ppm, or about 30 ppm of non-carrier copper in the precursor formulation. In still a further embodiment, the amount of non-carrier copper in the precursor formulation is about 5 ppm to about 30 ppm. In still a further embodiment, the amount of non-carrier copper in the precursor formulation is less than about 30 ppm.

### F. Buffer Solution

In one embodiment, the buffer solution used in the preparation of the bulk solution of the drug product is sodium acetate buffer, sodium acetate/gentisic acid buffer, sodium ascorbate buffer, sodium ascorbate/ethanol buffer, ammonium acetate buffer, ammonium acetate/gentisic acid buffer, ammonium ascorbate buffer, ammonium ascorbate/ethanol buffer, sodium phosphate/sodium diphosphate, sodium citrate/citric acid or any other appropriate buffer. In one embodiment, the concentration of gentisic acid in the buffer is from about 1 mg/mL to about 50 mg/mL, from about 1 mg/mL to about 30 mg/mL, from about 1 mg/mL to about 10 mg/mL, or from about 1 mg/mL to about 50 mg/mL. In yet another embodiment, the concentration of gentisic acid in the buffer is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 20 mg/mL, about 30 mg/mL, about 40 mg/mL, or about 50 mg/mL.

In another embodiment, the concentration of sodium acetate in the buffer is from about 10 mg/mL to about 100 mg/mL, from about 20 mg/mL to about 80 mg/mL, from about 30 mg/mL to about 60 mg/mL, or from about 40 mg/mL to about 60 mg/mL. In yet another embodiment, the concentration of sodium acetate in the buffer is about 10 mg/mL, about 20 mg/mL, about 30 mg/mL, about 40 mg/mL, about 45 mg/mL, about 50 mg/mL, about 54.8 mg/mL, about 55 mg/mL, about 60 mg/mL, about 70 mg/mL, about 80 mg/mL, about 90 mg/mL, or about 100 mg/mL.

In another embodiment, the buffer comprises about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL or about 100 mg/mL gentisic acid and about 10 mg/mL, about 20 mg/mL, about 30 mg/mL, about 40 mg/mL, about 45 mg/mL, about 50 mg/mL, about 54.8 mg/mL, about 55 mg/mL, about 60 mg/mL, about 70 mg/mL, about 80 mg/mL, about 90 mg/mL, or about 100 mg/mL of sodium acetate.

In one embodiment, the buffer is a solution containing between about 2 mg/mL to about 4 mg/mL gentisic acid and between about 0.25 M to about 0.4 M sodium acetate. In another embodiment, the buffer is a solution containing about 4 mg/mL of gentisic acid and about 55 mg/mL of sodium acetate. In another embodiment, the buffer solution has a pH of about 4.5 to about 5.5.

### G. Stabilizers

In one embodiment, the stabilizer is gentisic acid. In another embodiment, the stabilizer is ascorbate. However, any appropriate stabilizer may be used.

In another embodiment, more than one stabilizer is used. In another embodiment, one stabilizer, such as gentisic acid, is used during the radiolabeling process, and another stabilizer, such as sodium ascorbate or ascorbic acid, is used in the final formulated product.

In yet another embodiment, the stabilizer or a combination thereof is added to the reaction mixture in a concentration of about 1 mg/mL, 2 mg/mL, 3 mg/mL, 4 mg/mL, 5 mg/mL, 6 mg/mL, 7 mg/mL, 8 mg/mL, 9 mg/mL, about 10 mg/mL, 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

In one embodiment, sodium ascorbate is added to the reaction mixture in a concentration of about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

In another embodiment, ascorbic acid is added to the reaction mixture in a concentration of about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, or about 100 mg/mL.

### IV. Method of Detecting Prostate Cancer in a Human Subject

In one embodiment, the Cu-64 PSMA I&T products disclosed herein are administered to a human patient at a dosage of about 6.5 mCi, about 6.6 mCi, about 6.7 mCi, about 6.8 mCi, about 6.9 mCi, about 7.0 mCi, about 7.1 mCi, about 7.2 mCi, about 7.3 mCi, about 7.4 mCi, about 7.5 mCi, about 7.6 mCi, about 7.7 mCi, about 7.8 mCi, about 7.9 mCi, about 8.0 mCi, about 8.1 mCi, about 8.2 mCi, about 8.3 mCi, about 8.4 mCi, about 8.5 mCi, about 8.6 mCi, about 8.7 mCi, about 8.8 mCi, about 8.9 mCi, about 9.0 mCi, about 9.1 mCi, about 9.2 mCi, about 9.3 mCi, about 9.4 mCi, and about 9.5 mCi.

In another embodiment, the Cu-64 PSMA I&T products disclosed herein are administered to a human patient at a dosage of about 6.5 mCi to about 7.0 mCi, about 7.0 mCi to about 7.5 mCi, about 7.5 mCi to about 8.0 mCi, about 8.0 mCi to about 8.5 mCi, about 8.5 mCi to about 9.0 mCi, and about 9.0 mCi to about 9.5 mCi.

In another embodiment, the present invention is administered to a human patient at a dosage of about 6.5 m Ci to about 9.5 mCi.

In another embodiment, the present invention is administered and then imaged using a computerized tomography scan.

In another embodiment, the present invention is administered and then imaged using a positron emission tomography scan.

In one embodiment, the Cu-64 PSMA I&T products disclosed herein are administered to a human patient, and the patient is imaged at about 15 minutes, about 30 minutes, about 45 minutes, about 1 hour, about 1 hour and 15 minutes, about 1 hour and 30 minutes, about 1 hour and 45 minutes, about 2 hours, about 2 hours and 15 minutes, about 2 hours and 30 minutes, about 2 hours and 45 minutes, about 3 hours, about 3 hours and 15 minutes, about 3 hours and 30 minutes, about 3 hours and 45 minutes, about 4 hours, about 4 hours and 15 minutes, about 4 hours and 30 minutes, about 4 hours and 45 minutes, and about 5 hours post administration.

In yet another embodiment, the Cu-64 PSMA I&T products disclosed herein are administered to a human patient, and the patient is imaged from about 30 minutes to about 60 minutes, about 30 minutes to about 90 minutes, about 45 minutes to about 90 minutes, about 60 minutes to about 90 minutes, about 45 minutes to about 120 minutes, about 45 minutes to about 150 minutes, about 45 minutes to about 180 minutes, about 60 minutes to about 180 minutes, about 60 minutes to about 225 minutes, about 45 minutes to about 225 minutes, about 60 minutes to about 285 minutes, and about 60 minutes to about 300 minutes post administration.

In still another embodiment, the Cu-64 PSMA I&T products disclosed herein are administered to a human patient, and the patient is imaged at about 0.10 hours, about 0.20 hours, about 0.30 hours, about 0.40 hours, about 0.50 hours, about 0.60 hours, about 0.70 hours, about 0.80 hours, about 0.90 hours, about 1.00 hours, about 1.10 hours, about 1.20 hours, about 1.30 hours, about 1.40 hours, about 1.50 hours, about 1.60 hours, about 1.70 hours, about 1.80 hours, about 1.90 hours, about 2.00 hours, about 2.10 hours, about 2.20 hours, about 2.30 hours, about 2.40 hours, about 2.50 hours, about 2.60 hours, about 2.70 hours, about 2.80 hours, about 2.90 hours, about 3.00 hours, about 3.10 hours, about 3.20 hours, about 3.30 hours, about 3.40 hours, about 3.50 hours, about 3.60 hours, about 3.70 hours, about 3.80 hours, about 3.90 hours, about 4.00 hours, about 4.10 hours, about 4.20 hours, about 4.30 hours, about 4.40 hours, about 4.50 hours, about 4.60 hours, about 4.70 hours, about 4.80 hours, about 4.90 hours, and about 5.00 hours post administration.

In a further embodiment, the Cu-64 PSMA I&T products disclosed herein are administered to a human patient, and the patient is imaged at about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes, about 180 minutes, about 190 minutes, about 200 minutes, about 210 minutes, about 220 minutes, about 230 minutes, about 240 minutes, about 250 minutes, about 260 minutes, about 270 minutes, about 280 minutes, about 290 minutes, and about 300 minutes post administration.

In another embodiment, the Cu-64 PSMA I&T products disclosed herein are administered to a human patient, and the patient is imaged anywhere from about 45 minutes to about 4 hours and 30 minutes post administration. In still another embodiment, the Cu-64 PSMA I&T product disclosed herein is administered to a human patient, and the patient is imaged anywhere from about 1 hour to about 4 hours post administration.

In one embodiment, the region level CLR of the present method at about 1 hour ranged from about 70% to about 80%, about 71% to about 81%, about 72% to about 82%, about 73% to about 83%, about 74% to about 84%, about 75% to about 85%, about 76% to about 86%, about 77% to about 87%, about 78% to about 88%, about 79% to about 89%, about 80% to about 90%, about 81% to about 91%, about 82% to about 92%, about 83% to about 93%, about 84% to about 94%, about 85% to about 95%, about 86% to about 96%, about 87% to about 97%, about 88% to about 98%, about 89% to about 99%, and about 90% to about 100%. In yet another embodiment, the patient level CLR at about 1 hour ranged from about 70% to about 90%, about 70% to about 85%, and about 75% to about 90%.

In another embodiment, the region level CLR of the present method at about 4 hours ranged from about 74% to about 84%, about 75% to about 85%, about 76% to about 86%, about 77% to about 87%, about 78% to about 88%, about 79% to about 89%, about 80% to about 90%, about 81% to about 91%, about 82% to about 92%, about 83% to about 93%, about 84% to about 94%, about 85% to about 95%, about 86% to about 96%, about 87% to about 97%, about 88% to about 98%, about 89% to about 99%, and about 90% to about 100%. In yet another embodiment, the patient level CLR at about 4 hour ranged from about 70% to about 90%, about 70% to about 85%, and about 75% to about 90%.

In one embodiment, images obtained using the methods disclosed herein produced region level CLRs greater than about 60%. In yet another embodiment, images obtained using the methods disclosed herein produced region level CLRs greater than about 70%. In still another embodiment, images obtained using the methods disclosed herein produced region level CLRs greater than about 80%. In a further embodiment, images obtained using the methods disclosed herein produced region level CLRs greater than about 90%.

In another embodiment, the region level CLRs of the present method at 1 hour and at 4 hours, were about 15%, about 14%, about 13%, about 12%, and 11%, about 10%, about 9%, about 8%, about 7%, about 6%, about 5%, about 4%, about 3%, about 2%, and about 1% from each other.

In another embodiment, the patient level CDR at about 1 hour ranged from about 70% to about 77%, about 71% to about 78%, about 72% to about 79%, about 73% to about 80%, about 74% to about 81%, about 75% to about 82%, about 76% to about 83%, about 77% to about 84%, about 78% to about 85%, about 79% to about 86%, about 80% to about 87%, about 81% to about 88%, about 82% to about 89%, about 83% to about 90%, about 84% to about 91%, about 85% to about 92%, about 86% to about 93%, about 87% to about 94%, about 88% to about 95%, about 89% to about 96%, about 90% to about 97%, about 91% to about 98%, about 92% to about 99%, and about 93% to about 100%. In yet another embodiment, the patient level CDR at about 1 hour ranged from about 70% to about 90%, about 70% to about 85%, and about 75% to about 90%.

In another embodiment, the patient level CDR at about 4 hours ranged from about 72% to about 77%, about 73% to about 78%, about 74% to about 79%, about 75% to about 80%, about 76% to about 81%, about 77% to about 82%, about 78% to about 83%, about 79% to about 84%, about 80% to about 85%, about 81% to about 86%, about 82% to about 87%, about 83% to about 88%, about 84% to about 89%, about 85% to about 90%, about 86% to about 91%, about 87% to about 92%, about 88% to about 93%, about 89% to about 94%, about 90% to about 95%, about 91% to about 96%, about 92% to about 97%, about 93% to about 98%, about 94% to about 99%, about 95% to about 100%. In yet another embodiment, the patient level CDR at about 4 hour ranged from about 70% to about 90%, about 70% to about 85%, and about 75% to about 90%.

In one embodiment, images obtained using the methods disclosed herein produced patient level CDRs greater than about 60%. In yet another embodiment, images obtained using the methods disclosed herein produced patient level CDRs greater than about 70%. In still another embodiment, images obtained using the methods disclosed herein produced patient level CDRs greater than about 80%. In a further embodiment, images obtained using the methods disclosed herein produced patient level CDRs greater than about 90%.

In another embodiment, the patient level CDRs of the present method at 1 hour and at 4 hours, were about 15%, about 14%, about 13%, about 12%, and 11%, about 10%, about 9%, about 8%, about 7%, about 6%, about 5%, about 4%, about 3%, about 2%, and about 1% from each other.

In one embodiment, images obtained using the methods disclosed herein produced both region level CLRs and patient level CDRs greater than about 60%. In yet another embodiment, images obtained using the methods disclosed herein produced both region level CLRs and patient level CDRs greater than about 70%. In still another embodiment, images obtained using the methods disclosed herein produced both region level CLRs and patient level CDRs greater than about 80%. In a further embodiment, images obtained using the methods disclosed herein produced both region level CLRs and patient level CDRs greater than about 90%.

In another embodiment, the methods disclosed herein produced acceptable diagnostic images or AIQ at 1 hour and at 4 hours post administration of the Cu-64 PSMA I&T products disclosed herein to a human subject.

### Examples

### I. Example One: A Method of Detecting Prostate Cancer in a Human Subject Using a Cu-64 PSMA I&T Injection

### A. The Study Design

A study was conducted to evaluate the use of Cu-64 PSMA I&T injection in imaging human patients with recurrent metastatic prostate cancer. The primary objectives were to determine efficacy on region and patient level and to assess safety for use with humans. The secondary objective was to assess image quality or AIQ using CLR and CDR.

The study had two co-primary endpoints. First, region-level CLR defined as the percentage of regions containing at least one true PET positive lesion, with exactly localized correspondence between PET/CT imaging and the Composite Reference Standard regardless of any co-existent false positive findings, within the same region, out of all regions containing at least one PET positive finding. Second, patient-level CDR defined as the percentage of patients who have at least one true PET positive lesion, with exactly localized correspondence between PET imaging and the Composite Reference Standard, regardless of any co-existent false positive findings, out of all patients who are scanned.

Both endpoints were measured for 1 and 4 hours post injection using PET scans.

The study was powered to detect a CLR (detection rate) of greater than 60%. A sample size of 26 patients has an 80% power of detecting a true sensitivity of 85%. Key inclusion criteria included histologically proven prostate adenocarcinoma, prior radical prostatectomy, radiation therapy with curative intent, disease recurrence based on defined rise in PSA, and at least one extra prostatic site of disease suspected based on prior imaging or diagnosed by biopsy.

Patients were administered 7-9 mCi Cu-64 PSMA I&T injection and then PET and/or CT images were acquired for all patients starting at approximately 1 hour 15 minutes and 4 hours±30 minutes post Cu-64 PSMA I&T injection. Indeed, the images were collected from an approximate range of 45 minutes to 4 hours and 30 minutes post injection for all patients.

The PET and CT images were interpreted independently by three readers blinded to all patient information. Each patient study was assessed and scored for the detection of prostate cancer. Specifically, each reader categorized images as “Disease” or “No Disease” based only on tumor uptake of Cu-64 PSMA-I&T in each of the five regions: (1) prostate bed or prostate gland, (2) lymph nodes (pelvic), (3) lymph nodes (extra pelvic), (4) bone, and (5) viscera/soft tissue. Analysis of the reads were used for determination of the CLR and CDR by comparison to the Composite Reference Standard. The Composite Reference Standard was defined as local histopathology obtained within 60 days before or following Cu-64 PSMA I&T PET/CT, or, if histopathology was not available, anatomical correlation of conventional image findings (e.g., targeted MRI or CT or any FDA-approved PET PSMA agent) obtained within 60 days before or following Cu-64 PSMA I&T PET/CT.

A statistical threshold (with a lower bound of the 95% confidence interval) of 60% was prespecified for both CLR and CDR endpoints.

### B. Results of the Study

### Baseline Characteristics.

A total of 26 patients were enrolled in the human study. The mean PSA level prior to dosing was 54 ng/mL (SD 190) with a median of 2.77 ng/mL and a range of 0.30-790 ng/mL (min-max). The distribution of reference standard component for the primary endpoints were histopathology 0 patients (0%), CT scan 15 patients (32.61%), MRI 4 patients (8.70%), bone scan 11 patients (23.91%) and other PSMA PET scan 16 patients (34.78%). Fifteen patients had more than one reference standard modality performed.

The per-patient sensitivity and specificity were calculated and then averaged across all patients as shown in Table 3. In this evaluation, each of the patients were independent and were thus unique in the calculation. 19 patients had positive lesions on the Cu-64 PSMA I&T PET/CT scan while 7 patients had no lesions detected.

Table 3, which shows the overall summary of the per patient sensitivity and specificity ITI population, demonstrates a correct patient detection rate of 100.00% for all patients at both 1 and 4 hour images. It also shows a 95% confidence interval range of 82.35% to 100% on the high end and 59.04% to 100% on the low end.

The per patient specificity demonstrate that although all patients had verified BCR there was not significant reader bias towards PET scan classification as sick.

TABLE 3
Summary of Per Patient Sensitivity and Specificity ITI Population

| | | | Number of | Correct | Exact 95% |
|---|---|---|---|---|---|
| | | | Patients | Patient | Confidence |
| Parameter | Reader | Visit | with Truth | Detection Rate | Interval |
| Pet Patient | READER 1 | 1 Hour image | 19 | 100.00% | (82.35%, 100.00) |
| Sensitivity | | 4 Hour Image | 19 | 100.00% | (82.35%, 100.00) |
| | READER 2 | 1 Hour Image | 19 | 100.00% | (82.35%, 100.00) |
| | | 4 Hour Image | 19 | 100.00% | (82 35%, 100.00) |
| | READER 3 | 1 Hour Image | 19 | 100.00% | (82.35%, 100.00) |
| | | 4 Hour Image | 19 | 100.00% | (82.35%, 100.00) |
| Pa Patient | READER 1 | 1 Hour Image | 7 | 100.00% | (59.04%, 100.00) |
| Specificity | | 4 Hour Image | 7 | 100.00% | (59 04%, 100.00) |
| | READER 2 | 1 Hour Image | 7 | 100.00% | (59.04%, 100.00) |
| | | 4 Hour Image | 7 | 100.00% | (59.04%, 100.00) |
| | READER 3 | 1 Hour Image | 7 | 100.00% | (59.04%, 100.00) |
| | | 4 Hour Image | 7 | 103.00% | (59.04%, 100.00) |

The results were unexpected and exceeded original estimates and expectations. For example, the region level CLR and patient level CDR exceeded the prespecified threshold of 60% for all 3 readers at both 1 and 4 hours. Specifically, the region level CLR ranged from 76.67% (57.72-90.07%) to 86.67% (69.28-96.24%) at 1 hour and from 80.00% (61.43-92.29%) to 86.67% (69.28-96.24%) at 4 hours, as shown in Table 4. Similarly, the patient level CDR ranged from 77.19% (58.79-95.59%) to 84.21% (68.04-100.00%) at 1 hour and from 79.82% (61.55-98.10%) to 84.21% (68.04-100.00%) at 4 hours, as shown in Table 5. Accordingly, no numerical differences between endpoints at 1 and 4 hours were found.

TABLE 4
Summary of Primary Efficacy Endpoint - Correct Localization Rate (CLR)
(Primary Endpoint)
ITI Population

| | | | | Number of | | | |
|---|---|---|---|---|---|---|---|
| | | | Number of | Segments | Correct | Exact 95% | Exact p-value |
| | | | Segments | Correctly | Localization | Confidence | Ho:CDL = 60% |
| Parameter | Reader | Visit | Truth | Identified | Rate | Interval | Ha:CDL > 60% |
| Correct | READER 1 | 1 Hour | 30 | 26 | 86.67% | (69.28%, 96.24%) | 0.0015 |
| Localization | | Image | | | | | |
| Rate | | | | | | | |
| | | 4 Hour | 30 | 26 | 86.67% | (69.28%, 96.24%) | 0.0015 |
| | | Image | | | | | |
| | READER 2 | 1 Hour | 30 | 26 | 76.67% | (57.72%, 90.07%) | 0.0435 |
| | | Image | | | | | |
| | | 4 Hour | 30 | 24 | 80.00% | (61.43%, 92.29%) | 0.0172 |
| | | Image | | | | | |
| | READER 3 | 1 Hour | 30 | 24 | 80.00% | (61.43%, 92.29%) | 0.0172 |
| | | Image | | | | | |
| | | 4 Hour | 30 | 24 | 80.00% | (61.43%, 92.29%) | 0.0172 |
| | | Image | | | | | |

TABLE 5
Summary of Primary Efficacy Endpoint - Patient Level Correct Detection Rate (CDR)
(Primary Endpoint)
ITI Population

| | | | Number of | | | |
|---|---|---|---|---|---|---|
| | | | Patients with | Average Correct | Exact 95% | Exact p-value |
| | | | Positive | Detection Rate | Confidence | Ho:Sens = 60% |
| Parameter | Reader | Visit | Segments | (Standard Deviation) | Interval | Ha:Sens > 60% |
| Average | READER 1 | 1 Hour | 19 | 84.21% (33.552%) | (68.04%, 100.00%) | 0.0056 |
| Patient | | Image | | | | |
| CDR | | | | | | |
| | | 4 Hour | 19 | 84.21% (33.552%) | (68.04%, 100.00%) | 0.0056 |
| | | Image | | | | |
| | READER 2 | 1 Hour | 19 | 77.19% (38.172%) | (58.79%, 95.59%) | 0.0653 |
| | | Image | | | | |
| | | 4 Hour | 19 | 79.82% (37.916%) | (61.55%, 98.10%) | 0.0351 |
| | | Image | | | | |
| | READER 3 | 1 Hour | 19 | 80.70% (35.252%) | (63.71%, 97.69%) | 0.0197 |
| | | Image | | | | |
| | | 4 Hour | 19 | 80.70% (35.252%) | (63.71%, 97.69%) | 0.0197 |
| | | Image | | | | |

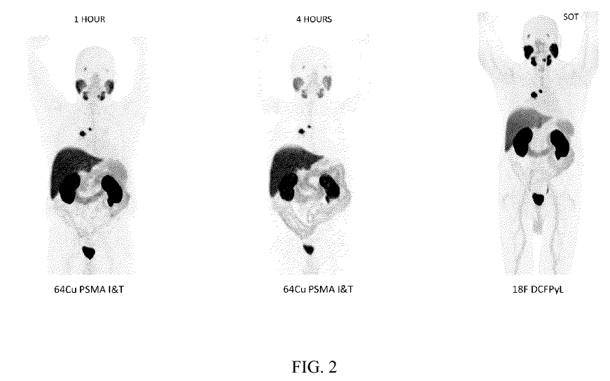

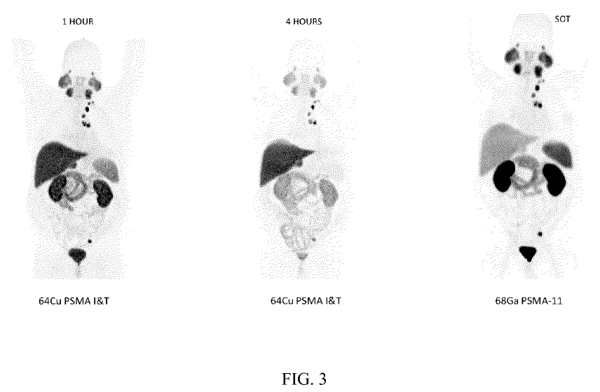

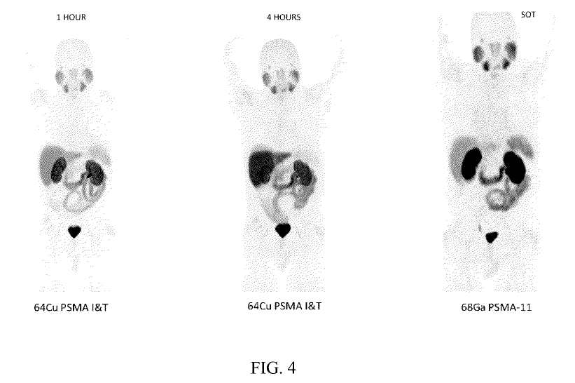

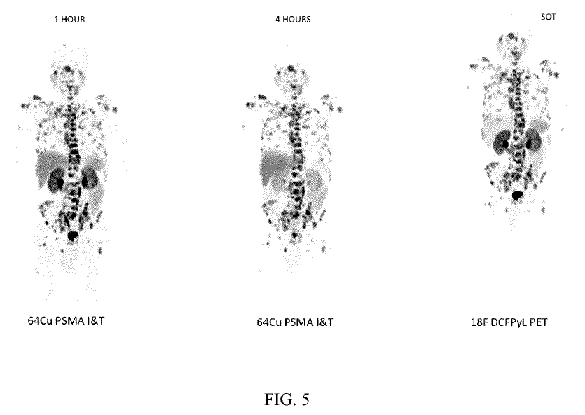

As Table 4 and Table 5 show, the CLR and CDR rates consistently retained AIQ from 1 hour±15 minutes to 4 hours±30 minutes post Cu-64 PSMA I&T injection. (See, e.g., FIGS. 2-5). In fact, not only did the CLR and CDR rates retain AIQ, in some instances the rates improved as the visit approached or elapsed the 4-hour mark. These unexpected results show that medical staff can accurately and effectively image patients up to and in excess of 4 hours post Cu-64 PSMA I&T injection. What's more, these results also indicate that CLR and CDR rates will consistency retain AIQ for imaging in excess of 4 hours and 30 minutes.

The successful primary efficacy endpoint CLR and CDR is further provided in Table 6 below:

TABLE 6
Primary Efficacy Endpoint CDR and CLR

| | Diagnostic performance | Reader 1 | Reader 2 | Reader3 |
|---|---|---|---|---|
| Level/region | measure | N = 26 | N = 26 | N = 26 |
| Patient level | Avg CDR % (SD) | 84.21 (33.552) | 77.19 (38.172) | 80.70 (35.252) |
| CDR (1 hour) | 95% C1 | 68.04, 100 | 58.79, 95.59 | 63.71, 97.69 |
| | P-value (H0: | 0.0056 | 0.0653 | 0.0197 |
| | CDR ≤ 60%) | | | |
| Patient level | Avg CDR % (SD) | 84.21 (33.552) | 79.82 (37.916) | 80.70 (35.252) |
| CDR (4 hours) | 95% C1 | 68.04, 100 | 61.55, 98.10 | 63.71, 97.69 |
| | P-value (H0: | 0.0056 | 0.0351 | 0.0197 |
| | CDR ≤ 60%) | | | |
| Region level | Avg CLR % | 86.67 | 76.67 | 80.00 |
| CLR (1 hour) | 95% C1 | 69.28, 96.24 | 57.72, 90.07 | 61.43, 92.29 |
| | P-value (H0: | 0.0015 | 0.0435 | 0.0172 |
| | CDR ≤ 60%) | | | |
| Region level | Avg CLR % | 86.67 | 80.00 | 80.00 |
| CLR (4 hours) | 95% C1 | 69.28, 96.24 | 61.43, 92.29 | 61.43, 92.29 |
| | P-value (H0: | 0.0015 | 0.0172 | 0.0172 |
| | CDR ≤ 60%) | | | |

HD tested using an exact one-sample binomial test with a one-sided a = 0.025
Patient-level CDR and region-level CLR exceeded the prespecified threshold of 60% for 2 of 3 readers at both 1 and 4 hours

The results are impacted by 16 patients having of other PSMA PET/CT as Composite Reference Standard offering a high sensitivity reference standard modality. Conventional imaging modalities are expected to be the predominant reference standard in phase 3 BCR trial. This less sensitive method will impact endpoints because of small lesions being denoted false positive that would be considered true positive if compared to a more sensitive reference standard. It is therefore likely that because of not allowing other PSMA PET as reference standard in phase 3 BCR trial, the CDR and CLR values will be lower.

### Secondary Efficacy Analyses.

Diagnostic quality was assessed for all scans using a 3-point scale and then percentages for each category were summarized for each reader and time point. No formal hypothesis testing was performed.

The independent readers assessed the image quality as acceptable at both 1 and 4 hours as shown in Table 7. In two patients, reader 2 found the image quality improving from questionable at 1 hour to acceptable at 4 hours suggesting a benefit of option for later images in selected patients.

TABLE 7
Summary of Image Quality

| | Image | Reader | Reader | Reader |
|---|---|---|---|---|
| Visit | Quality | 1 | 2 | 3 |
| 1 Hour | Inadequate | | | |
| Post | Questionable | | 2 (7.69%) | |
| Dose | Acceptable | 26 (100.00%) | 24 (92.31%) | 26 (100.00%) |
| 4 Hours | Inadequate | | | |
| Post | Questionable | | | |
| Dose | Acceptable | 26 (100.00%) | 26 (100.00%) | 26 (100.00%) |

Further, Table 7 shows that Readers 1 and 3 found perfect consistency in image quality between 1 and 4 hours post dose. Whereas, only Reader 2 found 2 instances of questionable image quality at 1 hour—yet all instances of image quality acceptable at 4 hours. These results show that the images are more similar than different at 1 and 4 hours. They also show a nice wash out of the kidneys and bladder at 4 hours. And the clearance of tracer from the bladder at 4 hours may be advantageous for diagnostic purposes. In sum, these results show that imaging can successfully be accomplished up to 4+ hours post dosage.

### C. Safety Analyses

### Analysis of Adverse Events.

Table 8 shows the total number of adverse events in the Phase 1 and 2 trials. Listing 9 and 10 shows the adverse event information and MedRA coding information.

A total of 4 adverse events were observed in 4 patients of the 30 patients dosed with 9 mCi Cu-64 PSMA I&T: dermatitis contact (1 (3.8%)), international normalized ratio (INR) increased (1 (3.8%)), hypomagnesaemia (1 (3.8%)), and claustrophobia (1 (3.8%)). All events were reported as not serious, of mild severity and not related to study product. No serious adverse events were attributed to Cu-64 PSMA I&T injection.

TABLE 8
Summary of Adverse Events by MedDRA System Organ Class and Preferred Term

| | Phase I | Phase I | Phase I | Phase II | Combined | | All Doses |
|---|---|---|---|---|---|---|---|
| MedDRA System Organ Class | 5 mCi | 7 mCi | 9 mCi | 9 mCi | 9 mCi | | Combined |
| Preferred Tenn | (N = 4) | (N = 4) | (N = 4) | (N = 26) | (N = 30) | | (N = 38) |
| Total Number of AE events | 2 | | 1 | 3 | 4 | | 6 |
| Subjects with at least one AE | 1 (25.0) | 0 | 1 (25.0) | 3 (11.5) | 4 | (13.3) | 5 (13.2) |
| General disorders and | 1 (25.0) | 0 | 0 | 0 | 0 | | 1 (2.6) |
| administration site conditio | | | | | | | |
| Feeling hot | 1 (25.0) | 0 | 0 | 0 | 0 | | 1 (2.6) |
| Immune system disorders | 0 | 0 | 1 (25.0) | 0 | 1 | (33) | 1 (2.6) |
| Dermatitis contact | 0 | 0 | 1 (25.0) | 0 | 1 | (3.3) | 1 (2.6) |
| Investigations | 0 | 0 | 0 | 1 (3.8) | 1 | (33) | 1 (2.6) |
| International normalized | 0 | 0 | 0 | 1 (3.8) | 1 | (33) | 1 (2.6) |
| ratio increased | | | | | | | |
| Metabolism and | 0 | 0 | 0 | 1 (3.8) | 1 | (33) | 1 (2.6) |
| nutrition disorders | | | | | | | |
| Hypomagnesaemia | 0 | 0 | 0 | 1 (3.8) | 1 | (33) | 1 (2.6) |
| Psychiatric disorders | 0 | 0 | 0 | 1 (3.8) | 1 | (33) | 1 (2.6) |
| Claustrophobia | 0 | 0 | 0 | 1 (3.8) | 1 | (33) | 1 (2.6) |

| | | | | | | |
|---|---|---|---|---|---|---|
| Skin and subcutaneous | 1 (25.0) | 0 | 0 | 0 | 0 | 1 (2.6) |
| tissue disorders | | | | | | |
| Hyperhidrosis | 1 (25.0) | 0 | 0 | 0 | 0 | 1 (2.6) |

Listing 9
Listing of Adverse Event Information

| Subject | | Adverse | Start | End | | | | | Action |
|---|---|---|---|---|---|---|---|---|---|
| ID | Treatment | Event Term | Date | Date | Serious | Outcome | Severity | Relationship | Taken |
| 01-002 | Phase | Allergic Reaction | 17 May | 23 May | No | Resolved | Mild | Not | None |
| | I -9 mCi | ECG adhesive | 2022 | 2022 | | | | Related | |
| 04-201 | Phase | International | 19 Jan. | 18 Apr. | No | Recovered | Mild | Not | None |
| | U.S mCi | Normalized | 2023 | 2023 | | | | Related | |
| | | Ratio Increased | | | | | | | |
| 05-002 | Phase | Hot | 7 Apr. | 7 Apr. | No | Resolved | Mild | Not | None |
| | I -5 mCi | | 2022 | 2022 | | | | Related | |
| | | Sweaty | 7 Apr. | 7 Apr. | No | Resolved | Mild | Not | None |
| | | | 2022 | 2022 | | | | Related | |
| 09-204 | Phase | HYPOMAGNESEMIA | 16 Mar. | Ongoing | No | Not | Mild | Not | Concomitant |
| | II-9 mCi | | 2023 | | | Recovered | | Related | Med |
| 09-206 | Phase | INTERMITTENT | 4 May | Ongoing | No | Not | Mild | Not | None |
| | II-9 mCi | CLAUSTROPHOBIA | 2023 | | | Recovered | | Related | |

Listing 10
Listing of Adverse Event MedRA Coding Information

| Subject | | Adverse | System | Preferred |
|---|---|---|---|---|
| ID | Treatment | Event Term | Organ Class | Term |
| 01-002 | Phase | Allergic Reaction | Immune system disorders | Dermatitis contact |
| | I -9 mCi | ECG adhesive | | |
| 04-201 | Phase | International | Investigations | International normalized |
| | II-9 mCi | Normalized | | ratio increased |
| | | Ratio Increased | | |
| 05-002 | Phase | Hot | General disorders and | Feeling hot |
| | I -5 mCi | | administration site conditio | |
| | | Sweaty | Skin and subcutaneous | Hyperhidrosis |
| | | | tissue disorders | |
| 09-204 | Phase | HYPOMAGNESEMIA | Metabolism and | Hypomagnesaemia |
| | II-9 mCi | | nutrition disorders | |
| 09-206 | Phase | INTERMITTENT | Psychiatric disorders | Claustrophobia |
| | II-9 mCi | CLAUSTROPHOBIA | | |

### D. Conclusion

Both co-primary endpoints of CDR and CLR were met, and favorable safety profile demonstrated. No numerical differences were found in efficacy endpoints between 1 and 4 hours and image quality were acceptable at both time points.

### II. Example Two: A Method of Detecting Prostate Cancer in a Human Subject Using a Cu-64 PSMA I&T Injection—Radiation Dosimetry Quantifications for PET/CT Imaging of Cu64 PSMA I&T Injection

### A. The Study Design

An open-label Phase 1/2 study was conducted to evaluate copper Cu-64 PSMA I&T injection for PET/CT imaging in patients with biochemical, recurrent PC after radical prostatectomy or radiation therapy. The Phase 1 portion assessed safety, determined radiation dosimetry estimates, and acceptable dose of copper Cu-64 PSMA I&T for obtaining diagnostic quality PET/CT images. The Phase 2 portion determined the CLR and CDR of copper Cu-64 PSMA I&T PET based on the selected dose from Phase 1 and further assessed safety post injection.

### Phase 1

The Phase 1 portion of the study included a total of 12 patients with biochemical, recurrent PC after prostatectomy or radiation therapy. Patients were randomized into 3 groups of 4 patients. Patients in a given group received a single intravenous dose of 5, 7, or 9 mCi (±10%) of copper Cu-64 PSMA I&T. Whole-body PET/CT images were acquired at 1 hour (±15 minutes), 4 hours (±30 minutes), and 24 hours (±2.0 hours) post injection.

Safety was assessed in all patients following administration of copper Cu-64 PSMA I&T injection through collection of AE reporting, laboratory assessments, vital signs, and ECG.

Radiation dosimetry estimates for normal organs were determined for copper Cu-64 PSMA I&T based on biodistribution collected from the 12 patients enrolled in the trial. Whole-body PET/CT images acquired at 1 hour (±15 minutes), 4 hours (±30 minutes), and 24 hours (±2.0 hours) post injection were used together with blood samples collected prior to and 1, 2, 4, and 24 hours post injection and total voided urine samples collected for 0 to 1-hour and 1 to 4-hour intervals post injection.

The 1-hour and 4-hour PET/CT images were evaluated independently by 3 readers blinded to all patient information, dose administration, and times of acquisition to assess the quality of the images (24 PET/CT image sets total). In addition, readers performed all assessments in one sitting with images scrambled such that the 1- and 4-hour imaging from the same patient was reviewed sequentially. The imaging protocol, scanner qualification, and imaging time/bed position were identical at 1, 4 and 24 hours and kept constant regardless of dose. The assessments were used together with dose reduction modeling to determine an acceptable dose to be administered for the Phase 2 portion of the study defined as the lowest copper Cu-64 PSMA I&T dose that yielded the most clinically relevant diagnostic image quality based on expert consensus review of all measurements and overall image quality.

The first-in-human Phase 1 portion was designed to illustrate and provide supporting evidence demonstrating the expected consequence of 5, 7, and 9 mCi injected doses of copper Cu-64 PSMA I&T PET/CT scans with the intention of determining a clinically relevant radioactive dose that takes into account the safety and image quality of the product. The dose range (5, 7, or 9 mCi) was chosen as a starting point based on several factors including first-hand experience with 64Cu-Dotatate studies demonstrating the ineffectiveness at lower radioactivity as well as literature demonstrating the efficacy of 18F-DCFPyl at a dosage of 9 mCi and 3-7 mCi for 68Ga PSMA-11. As the positron yield of Cu-64 (approximately 18%) is lower than F-18 (97%) and Ga-68 (88%), the higher range was chosen as a starting point to equate efficacy with other products with the same molecular target.

### Phase 2

The Phase 2 portion of the study included a separate group of 26 patients with biochemical recurrent PC. The dose for the Phase 2 portion of the study was 9 mCi with an acceptable range of 7 to 9 mCi. PET/CT images were acquired for all patients at 1 hour (±15 minutes) and 4 hours (±0.5 hours) post injection.

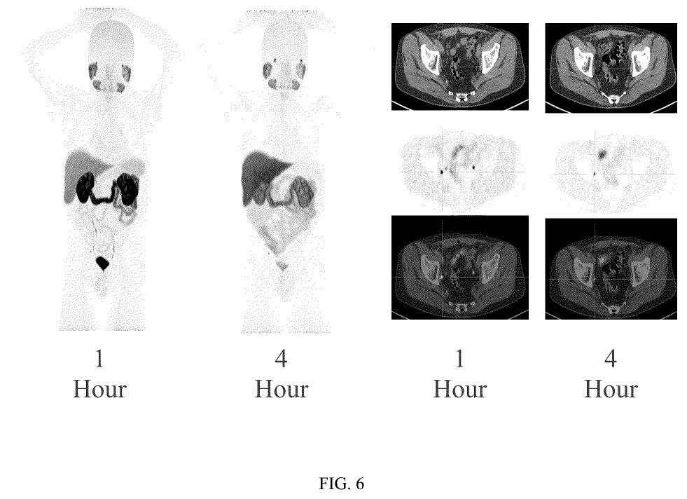

The PET/CT images were interpreted independently by 3 readers blinded to all patient information. Each patient was assessed and scored for the detection of prostate cancer lesions. Specifically, each reader categorized images as “Disease” or “No Disease” based only on tumor uptake of copper Cu-64 PSMA I&T in each of 5 regions: (1) prostate bed or prostate gland, (2) lymph nodes (pelvic), (3) lymph nodes (extra pelvic), (4) bone, and (5) viscera/soft tissue. Analysis of the reads was used for determination of the CLR and CDR for 1 hour and 4 hours post-injection imaging of copper Cu-64 PSMA I&T PET/CT by comparison to a composite reference standard collected for each enrolled patient. The composite reference standard was local histopathology obtained from PC surgery or biopsy obtained within 60 days before or after copper Cu-64 PSMA I&T PET/CT scans, or if histopathology was not available, anatomical correlation of conventional image findings (e.g., targeted MRI or CT) or any FDA-approved PSMA PET agent that were obtained within 60 days before or after copper Cu-64 PSMA I&T PET/CT scans. See, e.g., FIG. 6.

The Phase 2 portion assessed safety after copper Cu-64 PSMA I&T injection through collection of AEs 24 hours post-injection, lab assessments, vital signs, and ECGs.

The Phase 2 portion of the study was designed to establish a baseline diagnostic performance of copper Cu-64 PSMA I&T in a relevant PC patient population of the radioactive dose determined to have the most clinically relevant efficacy while also having a favorable safety profile. The Phase 2 study aimed to determine if the IP could effectively detect and localize PSMA-positive lesions. Additionally, the design sought to determine if the longer half-life of Cu-64 yielded clinically-relevant image quality and lesion detectability with a prolonged post-administration imaging start time by assessing images at 1 and 4 hours after IP injection.

### B. Image Quality Quantification

### Phase 1 Results

The Quantified Image Quality Scores (“QIQS”) for lesion evaluation and detection consistently demonstrated above average results across 5 mCi (scoring 1.72 QIQS), 7 mCi (scoring 1.81 QIQS) and 9 mCi (scoring 1.95 QIQS). These surprising results can be further shown in Table 9 below:

TABLE 9
Independent Reader Assessment of Lesion Evaluation Quality of 4-Hour Scans Based on
Number of Lesions Visible and Image Quality Score of 5, 7 and 9 mCi Scans (Mean [SD])

| | Lesion Evaluation Quality per Dose Group | | | | | |
|---|---|---|---|---|---|---|
| | 5 mCi | | 7 mCi | | 9 mCi | |
| | | Image | | Image | | Image |
| | Number of | Quality | Number of | Quality | Number of | Quality |
| Reader | Lesions | Score | Lesions | Score | Lesions | Score |
| Reader 1 | 19 | 2.00 (0.00) | 15 | 2.00 (0.00) | 12 | 2.00 (0.00) |
| Reader 2 | 17 | 2.00 (0.00) | 13 | 1.77 (0.44) | 13 | 2.00 (0.00) |
| Reader 3 | 19 | 1.16 (0.50) | 12 | 1.67 (0.65) | 13 | 1.85 (0.38) |
| Average | | 1.72 (0.49) | | 1.81 (0.17) | | 1.95 (0.09) |
| image quality | | | | | | |
| score-4 hours | | | | | | |

Source: Table 14.2.6

As shown in Table 9, a 5 mCi dose produced an Average QIQS (“AQIQS”) of 1.72—with a lower threshold scoring 1.16 and two perfect scores reaching 2.00 (standard deviation 0.49 on average). These results improved with increased dosages. For example, 7 mCi experienced a significant AQIQS improvement with 1.81 AQIQS, with a lower threshold reaching 1.67 QIQS and an upper threshold holding at 2.00 QIQS. Indeed, at 9 mCi, the QIQS were nearly perfect (with only 0.09 standard deviation for AQIQS). What is more, the benefit of these results are further enhanced by the fact that Cu64 produces increased image contrast and detection of smaller target areas (e.g., 1 cm) due to its long half life of 12.7 hours (as compared to the prior art, e.g., Ga-68).

Thus, a quantification of said study shows a clear relationship between increased mCi levels and increased QIQS scores for lesion evaluation and detection.

A second breakdown of the QIQS on an hourly level additionally demonstrates the surprising results of attaining clear imaging scores for lesion evaluation and detection at 4 hours post dose:

TABLE 10
Independent Reader Assessment of General Image Quality of 5, 7
or 9 mCi Scans Using Standard Image Score Definition (Mean [SD])

| | | General Image Quality Per Dose Group | | |
|---|---|---|---|---|
| | | 5 mCi | 7 mCi | 9 mCi |
| Reader/Timepoint | | (n = 4) | (n = 4) | (u = 4) |
| Reader 1 | 1 hour | 2.00 (0.00) | 2.00 (0.00) | 1.75 (0.50) |
| | 4 hours | 1.75 (0.50) | 2.00 (0.00) | 1.75 (0.50) |
| Reader 2 | 1 hour | 2.00 (0.00) | 1.00 (0.82) | 1.75 (0.50) |
| | 4 hours | 2.00 (0.00) | 1.50 (0.58) | 2.00 (0.00) |
| Reader 3 | 1 hour | 1.00 (0.00) | 1.25 (0.50) | 1.50 (0.58) |
| | 4 hours | 1.00 (0.82) | 1.75 (0.50) | 1.50 (0.58) |

| | | | |
|---|---|---|---|
| Average general image | 1.63 (0.58) | 1.58 (0.58) | 1.71 (0.46) |
| quality across timepoints | | | |

Indeed, as shown in Table 10, QIQS at 4 hours never dropped below 1.00 (with a standard deviation ranging from 0.82 to 0.50). Further, QIQS at 4 hours also repeatedly scored perfect 2.00 at 9 mCi, 7 mCi, and, most surprisingly, 5 mCi.

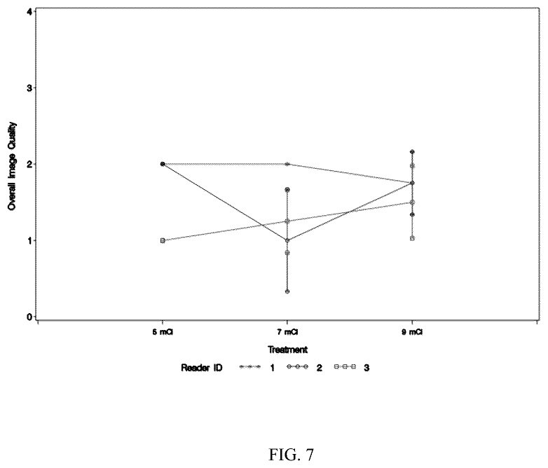

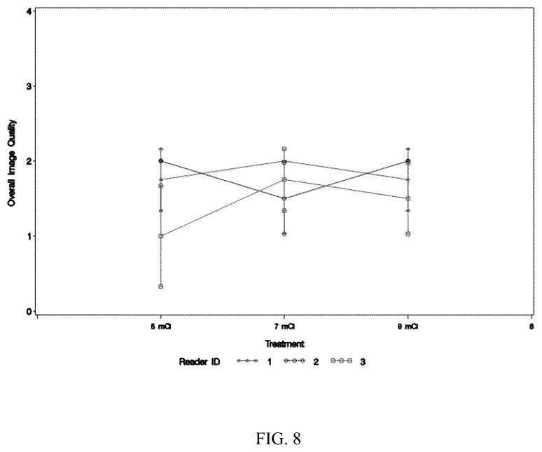

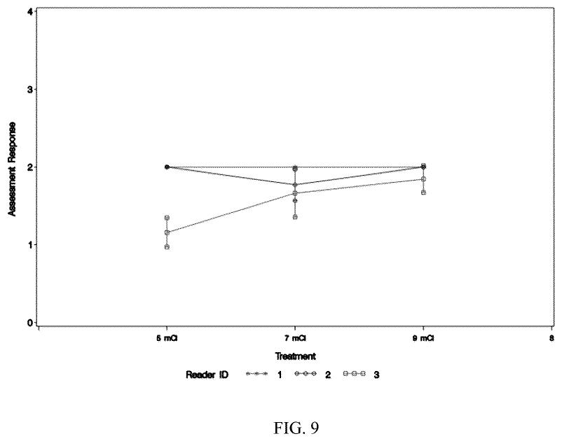

These quantifications show treatment availability possibilities at 4 hours for 5 mCi, 7 mCi, and 9 mCi. They also show an increased flexibility for treatment options, thereby increases the chances of success, as shown in FIGS. 7-9.

FIGS. 7-9 show that the treatment availability window is not exclusive to higher doses (e.g., 9 mCi) nor shorter times after dose (e.g., 1 hour); but, in fact, the opposite: that lower doses (e.g., 5 mCi) at longer times after dose (e.g., 4+ hours) produce satisfactory, if not exemplary results for PET and CT imaging.

Percent injected activity as a function of time was determined for the quantified organs listed below. On average, the single organ that showed the largest peak uptakes was the liver with approximately 30% of the injected activity at T3. Table 11 shows the percent injected activity summary statistics for all subjects in this analysis.

TABLE 11
Mean % Injected Dose vs Time (h)

| | Timepoint: | | |
|---|---|---|---|
| Source Organ | T1 | T2 | T3 |
| Stomach Wall | 0.52% | 0.51% | 0.42% |
| GI Total Contents | 10% | 10% | 12% |
| Heart wall | 1.2% | 0.84% | 0.96% |
| Kidneys | 8.6% | 5.0% | 2.1% |
| Lacrimal Glands | 0.023% | 0.024% | 0.015% |
| Liver | 19% | 25% | 30% |
| Pancreas | 0.35% | 0.39% | 0.28% |
| Pituitary | 0.0021% | 0.0019% | 0.0017% |
| Prostate | 0.036% | 0.031% | 0.021% |
| Salivary glands | 1.1% | 0.95% | 0.40% |
| Red Marrow | 1.7% | 1.4% | 1.5% |
| Spleen | 1.5% | 0.54% | 0.29% |
| Testes | 0.11% | 0.10% | 0.065% |
| Thyroid | 0.031% | 0.019% | 0.016% |
| Remainder | 52% | 42% | 33% |
| Whole Body | 96% | 87% | 81% |

| Timepoint: | T1 | T2 | T3 | T4 |
|---|---|---|---|---|
| Blood | 5.8% | 4.2% | 3.2% | 4.4% |

| Void Interval: | 0-1 hours | 1-4 hours |
|---|---|---|
| Urine Excretion | 4.4% | 6.2% |

A summary of normalized number of disintegrations statistics for subjects in this analysis are given in Table 12. The largest mean normalized organ number of disintegrations for the subjects were for liver (5.0 h), and kidney (0.69 h).

TABLE 12
Summary Statistics Normalized Number of Disintegrations (hours)

| | Mean | Min | Max | SD | CV |
|---|---|---|---|---|---|
| Stomach Wall | 0.083 | 0.041 | 0.18 | 0.044 | 53% |
| Heart wall | 0.17 | 0.14 | 0.21 | 0.023 | 13% |
| Kidneys | 0.69 | 0.35 | 0.86 | 0.15 | 22% |
| Lacrimal | 0.0033 | 0.0018 | 0.0077 | 0.0016 | 49% |
| Glands | | | | | |
| Liver | 5.0 | 4.0 | 5.7 | 0.53 | 11% |
| Pancreas | 0.058 | 0.032 | 0.11 | 0.021 | 37% |
| Pituitary | 0.00033 | 0.00023 | 0.00050 | 0.000089 | 27% |
| Prostate | 0.0047 | 0.00081 | 0.017 | 0.0053 | 113% |
| Salivary glands | 0.12 | 0.058 | 0.21 | 0.042 | 36% |
| Red Marrow | 0.28 | 0.10 | 0.38 | 0.085 | 31% |
| Spleen | 0.096 | 0.036 | 0.35 | 0.083 | 86% |
| Testes | 0.015 | 0.0027 | 0.038 | 0.0090 | 61% |
| Thyroid | 0.0035 | 0.0018 | 0.0074 | 0.0018 | 52% |
| Remainder | 6.9 | 5.9 | 8.3 | 0.72 | 10% |
| Whole Body | 15 | 14 | 16 | 0.73 | 4.8% |

Estimated dose summary statistics in mGy/MBq are shown in Table 13 for all subjects in this analysis. On average, the organs receiving the largest absorbed doses were the liver and left colon at 0.25 mGy/MBq respectively, followed by the right colon at 0.22 mGy/MBq. The kidneys received an average dose of 0.19 mGy/MBq. The red marrow received an average dose of 0.023 mGy/MBq. The average Effective Dose was 0.061 mGy/MBq.

TABLE 13
Cu 64 PSMA I&T Dose Estimates Summary Statistics (mGy/MBq)

| | Mean | Min | Max | SD | CV |
|---|---|---|---|---|---|
| Adrenals | 0.045 | 0.042 | 0.049 | 0.0024 | 5.4% |
| Brain | 0.011 | 0.0093 | 0.013 | 0.0010 | 9.9% |
| Esophagus | 0.020 | 0.019 | 0.022 | 0.0011 | 5.3% |
| Eyes | 0.010 | 0.0091 | 0.012 | 0.0010 | 9.9% |
| Gallbladder Wall | 0.051 | 0.047 | 0.056 | 0.0029 | 5.7% |
| Left colon | 0.25 | 0.19 | 0.39 | 0.051 | 21% |
| Small Intestine | 0.067 | 0.054 | 0.10 | 0.011 | 17% |
| Stomach Wall | 0.063 | 0.042 | 0.11 | 0.022 | 35% |
| Right colon | 0.22 | 0.17 | 0.34 | 0.044 | 20% |
| Rectum | 0.15 | 0.12 | 0.24 | 0.031 | 20% |
| Heart Wall | 0.055 | 0.048 | 0.065 | 0.0056 | 10% |
| Kidneys | 0.19 | 0.11 | 0.24 | 0.038 | 20% |
| Lacrimal Glands | 0.16 | 0.087 | 0.37 | 0.077 | 49% |
| Liver | 0.25 | 0.21 | 0.29 | 0.026 | 10% |
| Lungs | 0.018 | 0.017 | 0.020 | 0.0011 | 5.7% |
| Pituitary | 0.037 | 0.026 | 0.056 | 0.010 | 27% |
| Pancreas | 0.054 | 0.040 | 0.080 | 0.011 | 21% |
| Prostate | 0.027 | 0.014 | 0.085 | 0.019 | 72% |
| Salivary Glands | 0.12 | 0.062 | 0.21 | 0.041 | 34% |
| Red Marrow | 0.023 | 0.016 | 0.027 | 0.0032 | 14% |
| Osteogenic Cells | 0.018 | 0.014 | 0.021 | 0.0022 | 12% |
| Spleen | 0.048 | 0.030 | 0.087 | 0.015 | 32% |
| Testes | 0.035 | 0.010 | 0.086 | 0.019 | 54% |
| Thymus | 0.015 | 0.014 | 0.017 | 0.0011 | 7.4% |
| Thyroid | 0.015 | 0.0095 | 0.020 | 0.0032 | 21% |
| Urinary Bladder Wall | 0.074 | 0.051 | 0.11 | 0.018 | 24% |
| Total Body | 0.022 | 0.020 | 0.023 | 0.0010 | 4.7% |
| Effective Dose | 0.061 | 0.054 | 0.079 | 0.0067 | 11% |

This analysis was performed with the objective of estimation of radiation dosimetry of Cu-64 PSMA I&T based on bio-distribution determined using PET image data collected in twelve human subjects. On average across the 12 patients, the sum of the unnormalized activity recovery from the whole body PET quantification and urinary excretion was excellent, at 100.9% of the injected activity at the first imaging time. The maximum deviation was 13%. Activities were normalized to 100% based on the first imaging time to prevent over or under estimation of absorbed dose.

On average, the organs receiving the largest absorbed doses were the liver and left colon at 0.25 mGy/MBq respectively, followed by the right colon at 0.22 mGy/MBq. The kidneys received an average dose of 0.19 mGy/MBq. The red marrow received an average dose of 0.023 mGy/MBq. The average Effective Dose was 0.061 mGy/MBq.

### Phase 2 Results

The CLR and CDR primary endpoints substantiate the veracity of these quantifications by consistently hitting high rates with exemplary confidence intervals. For example, the 4 hour CLR never dropped below 80% CLR and also never dropped below 61.43% confidence interval as shown in Table 14:

TABLE 14
Summary of Region Level Correct Localization Rate - IME Population

| | | | | Number of | | | |
|---|---|---|---|---|---|---|---|
| | | | Number of | Lesions | Current | Exact 95% | Exact p-value |
| | | | Lesions | Correctly | Localization | Confidence | H_{O}:CLR = 60% |
| Parameter | Reader | Visit | Truth | Identified | Rate | Interval | H_{A}:CLR > 60% |
| Correct | READER 1 | 1 Hour | 30 | 26 | 86.67% | (69.28%, 96.24%) | 0.0015 |
| Localization | | Image | | | | | |
| Rate | | 4 Hour | 30 | 26 | 86.67% | (69.28%, 96.24%) | 0.0015 |
| | | Image | | | | | |
| | READER 2 | 1 Hour | 30 | 23 | 7667% | (57.72%, 90.07%) | 0.0435 |
| | | Image | | | | | |
| | | 4 Hour | 30 | 24 | 80.00% | (61.43%, 92.29%) | 0.0172 |
| | | Image | | | | | |
| | READER 3 | 1 Hour | 30 | 24 | 80.110% | (61.43%, 92.29%) | 0.0172 |
| | | Image | | | | | |
| | | 4 Hour | 30 | 24 | 80.00% | (61.43%, 92.29%) | 0.0172 |
| | | Image | | | | | |

Abbreviation:
CLR, correct localization rate

Similarly surprising were the CDR quantifications also showing no CDR below 79.82% at 4 hours after dose. The confidence intervals were also impressive, showing no value lower than 58.79%. These calculations are represented below in Table 15:

TABLE 15
Patient Level Correct Detection Rate - IME Population

| | | | Number of | | | |
|---|---|---|---|---|---|---|
| | | | Patients | | | |
| | | | with | Average Correct | Exact 95% | Exact p-value |
| | | | Positive | Detection Rate | Confidence | H_{O}:Sens = 60% |
| Parameter | Reader | Visit | Lesions | (Standard Deviation) | Interval | H_{A}:Sens > 60% |
| Average | READER 1 | 1 Hour | 19 | 84.21% (33.552%) | (68.04%, 100.00%) | 0.0056 |
| Patient | | Image | | | | |
| CDR | | 4 Hour | 19 | 84.21% (33.552%) | (68.04%, 104:IMV) | 0.0056 |
| | | Image | | | | |
| | READER 2 | 1 Hour | 19 | 77.19% (38.172%) | (58.79%, 95.59%) | 0.0653 |
| | | Image | | | | |
| | | 4 Hour | 19 | 79.82% (37.916%) | (61.55%, 98.10%) | 0.0351 |
| | | Image | | | | |
| | READER 3 | 1 Hour | 19 | 80.70% (35.252%) | (63.71%, 97.69%) | 0.0197 |
| | | Image | | | | |
| | | 4 Hour | 19 | 80.70% (35.252%) | (63.71%, 97.69%) | 0.0197 |
| | | Image | | | | |

Abbreviation:
CDR, correct detection rate

In addition to these quantifications for arriving at the target PET/CT imaging levels, a series of calculations may be employed to ensure sufficient QIQS for lesion evaluation and detection. These are Activity Quantification, Normalization, and Source Organ Time-Activity.

First, calculating Activity Quantification in PET Images utilizing the following Equation One:

$${A}_{PET} = [ {A}_{PET} ] {V}_{VOI}$$

$$Where :$$

$$\begin{matrix}{A}_{PET} = Total Activity in Organ ( kBq ) \\ [ {A}_{PET} ] = Mean Activity Concentration in Organ ( kBq / mL ) \\ {V}_{VOI} = Volume of Volume of Interest ( mL )\end{matrix}$$

Use of this equation can calculate organ/tissue and whole body activity concentration levels.

Second, Normalization of PET Quantification utilizing a normalization factor to decay values using urine activity utilizing the following Equation Two:

$$NF = \frac{TBA}{100 - UA}$$

$$Where :$$

$$\begin{matrix}NF = Normalization Factor \\ TBA = Total Body Activity \\ UA = Urine Activity\end{matrix}$$

A normalization factor can be determined and applied to the decay corrected % IA values by assuming 100% quantification of the total body activity at the first time point and assuming accurate urine activity assay according to Equation Two.

Third, calculating Source Organ Time-Activity Bio-Kinetic Modeling using bio-distribution data for the listed organs utilizing the following Equation Three:

$$F ( t ) = {\sum }_{i} {f}_{i} {e}^{- {λ}_{i} t}$$

$$Where :$$

$$\begin{matrix}{f}_{i} = Fractional dose model parameter i ( determined in the fitting process ) \\ {λ}_{i} = Retention rate model parameter i ( determined in the fitting process ) \\ F ( t ) = Fraction of the total injected activity at time t \\ t = Time post injection \\ i = The {i}^{th} exponential term\end{matrix}$$

The bio-distribution data for the listed organs and remainder tissues in the study may be determined using the image quantification methodology described above. Organ and tissue data may be decay corrected to fit in a least squares sense using non-linear regression with sums of exponentials of the form shown in Equation Three.

Once these data are fit, normalized number of disintegrations (residence times or AUCs) may be determined by integration of these empirically determined functions (sums of exponentials) from time equal zero to infinity, taking into account physical decay using Equations Four and Five:

$$τ = {\int }_{0}^{∞} {\sum }_{i} {f}_{i} {e}^{- {λ}_{i} t} = {\sum }_{i} \frac{{f}_{i}}{( {λ}_{i} + {λ}_{R} )}$$

$${λ}_{R} = \frac{ln ( 2 )}{{T}_{\frac{1}{2} R}}$$

$$Where :$$

$$\begin{matrix}{λ}_{R} = Radiological decay constant \\ {T}_{\frac{1}{2} R} = isotope half - life ( h )\end{matrix}$$

For organs exhibiting increasing uptake between the final 2 imaging timepoints, indefinite biological retention may be assumed beyond the final imaging time, such that the normalized number of disintegrations determined are based upon physical decay only.

### C. Safety Analyses

### Analysis of Adverse Events

Table 16 shows the total number of adverse events in the Phase I and 2 trials.

TABLE 16
Summary of Adverse Events by MedDRA System Organ Class and Preferred Term

| | Phase I | Phase I | Phase I | Phase II | Combined | |
|---|---|---|---|---|---|---|
| | 5 mCi | 7 mCi | 9 mCi | 9 mCi | 9 mCi | |
| | 64Cu- | 64Cu- | 64Cu- | 64Cu- | 64Cu- | All Doses |
| MedDRA System Organ Class | PSMA I&T | PSMA I&T | PSMA I&T | PSMA I&T | PSMA I&T | Combined |
| Preferred Term | (N = 4) | (N = 4) | (N = 4) | (N = 26) | (N = 30) | (N = 38) |
| Total Number of AE events | 2 | | 1 | 3 | 4 | 6 |
| Subjects with at least one AE | 1 (25.0) | 0 | 1 (25.0) | 3 (11.5) | 4 (13.3) | 5 (13.2) |
| General disorders and | 1 (25.0) | 0 | 0 | 0 | 0 | 1 (2.6) |
| administration site conditions | | | | | | |
| Feeling hot | 1 (25.0) | 0 | 0 | 0 | 0 | 1 (2.6) |
| Immune system disorders | 0 | 0 | 1 (25.0) | 0 | 1 (3.3) | 1 (2.6) |
| Dermatitis contact | 0 | 0 | 1 (25.0) | 0 | 1 (3.3) | 1 (2.6) |
| Investigations | 0 | 0 | 0 | 1 (3.8) | 1 (3.3) | 1 (2.6) |
| International normalized | 0 | 0 | 0 | 1 (3.8) | 1 (3.3) | 1 (2.6) |
| ratio increased | | | | | | |
| Metabolism and | 0 | 0 | 0 | 1 (3.8) | 1 (3.3) | 1 (2.6) |
| nutrition disorders | | | | | | |
| Hypomagnesemia | 0 | 0 | 0 | 1 (3.8) | 1 (3.3) | 1 (2.6) |
| Psychiatric disorders | 0 | 0 | 0 | 1 (3.8) | 1 (3.3) | 1 (2.6) |
| Claustrophobia | 0 | 0 | 0 | 1 (3.8) | 1 (3.3) | 1 (2.6) |
| Skin and subcutaneous | 1 (25.0) | 0 | 0 | 0 | 0 | 1 (2.6) |
| tissue disorders | | | | | | |
| Hyperhidrosis | 1 (25.0) | 0 | 0 | 0 | 0 | 1 (2.6) |

A total Overall, 5 (13.2%) patients (2 in Phase 1 and 3 in Phase 2) experienced at least 1 AE during the study (Table 16), with all AEs (total 6 events) deemed as mild in severity and not related to the IP (Listing 16.2.7.1). All AEs were resolved except for the events of hypomagnesemia and intermittent claustrophobia, which were still ongoing at study completion. No action was taken with the IP in response to the AEs (Listing 16.2.7.1). No serious adverse events were attributed to Cu-64 PSMA I&T injection.

### D. Conclusion

This first in human study demonstrated Cu-64 PSMA I&T injection is safe and well tolerated and yields clinically relevant, high-quality images at a dosage range of 7-9 mCi. The co-primary endpoints of CDR and CLR were successfully met demonstrating Cu-64 PSMA I&T as a potentially safe and effective radiopharmaceutical for diagnostic PET in men with biochemical recurrent PC.

The embodiments described herein are intended to be merely exemplary. Persons skilled in the art will understand that variations and modifications may be made without departing from the scope of the invention encompassed by the claims below.

What is claimed is:

1. A method of detecting prostate cancer in a patient in need thereof, comprising the steps of:
(a) administering to the patient about 5 mCi to about 9 mCi of a Cu-64 PSMA I&T composition, and
(b) imaging the patient between about 1 hour 15 minutes to about 4 hours 30 minutes post administration of the composition,
wherein the image is of acceptable image quality.

2. The method of claim 1, wherein the patient is administered about 8 mCi of the Cu-64 PSMA I&T composition.

3. The method of claim 1, wherein the patient is imaged using positron emission tomography.

4. The method of claim 1, wherein the patient is imaged at about 1 hour post administration of the composition.

5. The method of claim 1, wherein the patient is imaged at about 2 hours post administration of the composition.

6. The method of claim 1, wherein the patient is imaged at about 3 hours post administration of the composition.

7. The method of claim 1, wherein the patient is imaged at about 4 hours post administration of the composition.

8. The method of claim 4, wherein the correct detection rate was greater than 60%.

9. The method of claim 7, wherein the correct detection rate was greater than 60%

10. A method of detecting prostate cancer in a patient in need thereof, comprising the steps of:
(a) administering to the patient about 7 mCi to about 9 mCi of a Cu-64 PSMA I&T composition, and
(b) imaging the patient up to 4.5 hours after administration of the composition;
wherein the resulting image allows a reader to detect prostate cancer in the patient.

11. The method of claim 10, wherein the patient is administered about 8 mCi of the Cu-64 PSMA I&T composition.

12. The method of claim 10, wherein the patient is imaged using positron emission tomography.

13. The method of claim 10, wherein the patient is imaged at about 1 hour post administration of the composition.

14. The method of claim 10, wherein the patient is imaged at about 2 hours post administration of the composition.

15. The method of claim 10, wherein the patient is imaged at about 3 hours post administration of the composition.

16. The method of claim 10, wherein the patient is imaged at about 4 hours post administration of the composition

17. The method of claim 13, wherein the correct detection rate was greater than 60%.

18. The method of claim 16, wherein the correct detection rate was greater than 60%.

19. A method of detecting prostate cancer in a patient in need thereof, comprising the steps of:
(a) administering to the patient about 5 mCi to about 9 mCi of a Cu-64 PSMA I&T composition, and
(b) imaging the patient between about 1 hour to about 4 hours post administration of the composition,
wherein the resulting image does not produce a correct detection rate below 80%.

20. The method of claim 19, wherein the patient is administered about 8 mCi of the Cu-64 PSMA I&T composition.

21. The method of claim 19, wherein the patient is imaged using positron emission tomography.

22. The method of claim 19, wherein the patient is imaged at about 1 hour post administration of the composition.

23. The method of claim 19, wherein the patient is imaged at about 2 hours post administration of the composition.

24. The method of claim 19, wherein the patient is imaged at about 3 hours post administration of the composition.

25. The method of claim 19, wherein the patient is imaged at about 4 hours post administration of the composition.
